(19) United States
(12) Patent Application Publication
(10) Pub. No.: US 2002/0068346 A1
(43) Pub. Date: Jun. 6, 2002

(54) Modified inosine 5'-monophosphate dehydrogenase polypeptides and uses thereof

(76) Inventors: Stanley R. Krystek, Ringoes, NJ (US); Steven Sheriff, Princeton, NJ (US); Mark R. Witmer, Pennington, NJ (US); Diane L. Hollenbaugh, Newton, PA (US); Ning Yan, Plainsboro, NJ (US); Julie E. Mouravieff, Princeton, NJ (US); Howard M. Einspahr, Lawrenceville, NJ (US); Kevin Kish, New Brunswick, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/853,918

(22) Filed: May 10, 2001

Related U.S. Application Data
(60) Provisional application No. 60/203,448, filed on May 10, 2000.

(51) Int. Cl. C12N 9/04; C12P 19/40

(52) U.S. Cl. 435/190; 435/88

(57) ABSTRACT

The present invention provides isolated modified inosine 5′-monophosphate dehydrogenase (IMPDH) polypeptides. These modified IMPDH polypeptides comprise a substitute oligo-peptide that replaces the subdomain region. The modified polypeptides of the invention bind to an inhibitor, such as MPA, and/or exhibit functional activity of wild-type IMPDH holo-enzyme.

This application claims the benefit of the filing date of U.S. Ser. No. 60/203,448, filed May 10, 2000. The content of the foregoing application in its entirety is incorporated by reference herein.

Throughout this application, various publications are referenced. The disclosures of these publications are hereby incorporated by reference herein in their entireties.

### FIELD OF THE INVENTION

The present invention involves isolated modified inosine 5′-monophosphate dehydrogenase (IMPDH) polypeptides comprising substitute oligo-peptides that replace a subdomain region.

### BACKGROUND OF THE INVENTION

The enzyme inosine-5′-monophosphate dehydrogenase (IMPDH; EC 1.1.1.205) is involved in the de novo synthesis of guanosine nucleotides (Crabtree, G. W., and Henderson, J. F. 1971 Cancer Res. 31:985-991; Snyder, F. F. et al., 1972 Biochem. Pharmacol. 21:2351-2357; Weber, G., 1983 Acct. Chem. Res. 24:209-215). IMPDH catalyzes the oxidation of inosine-5′-monophosphate (IMP) to xanthosine-5′-monophosphate (XMP)(Jackson, R. C. et. al., 1975 Nature 256:331-333). The IMPDH enzyme follows an ordered Bi-Bi reaction sequence of substrate and cofactor binding and product release. First, IMP binds to IMPDH, followed by binding of the cofactor NAD, followed by reduction to NADH. The reduced NADH is then released followed by the product, XMP (Carr, S. F. et al., 1993 J. Biol. Chem. 268:27286-90; Holmes, E. W. et al., 1974 Biochem. Biophys. Acta. 364:209-217). IMPDH is a ubiquitous enzyme found in all prokaryotes and eukaryotes (Natsumeda, Y. and Carr, S. F., 1993 Ann. N.Y Acad. 696:88-93). The IMPDH enzyme from all species exists as a homo-tetramer, comprising four subunits of type I or II IMPDH polypeptide. Each subunit is an IMPDH polypeptide that has a larger catalytic core domain and a smaller subdomain, or flanking domain, that has no known function. Each subunit is bound by a potassium ion (Xiang, B., et al., 1996 J. Biol. Chem. 271:1435-1440) which may organize the tetramer around the active site (Sintchak, M. D., et al., 1996 Cell 85:921-930). The amino acid sequences of prokaryotic IMPDH polypeptides share 30-40% sequence identity with human IMPDH (Natsumeda, Y. and Carr, S. F. 1993 supra). The amino acid sequence of the subdomain exhibits substantial variation among IMPDH from different species.

Two isoforms of human IMPDH, designated type I and type II, have been identified and sequenced (Collart, F. R. and Hubermann, E. 1988 J. Biol. Chem. 263:15769-15772; Natsumeda, Y., et al. 1990 J. Biol. Chem. 265:5292-5295). Both type I and II are 514 amino acid residues in length, and they share 84% sequence identity. Variations in the sequences of the type I and type II IMPDH molecules have also been disclosed. A nucleotide and amino acid sequence of a wild type human IMPDH type II is disclosed in Natsumeda, Y., et al. 1990 J. Biol. Chem. 265:5292-5295; Collart, F. R. and Hubermann, E. 1988 J. Biol. Chem. 263:15769-15772; and U.S. Pat. No. 5,665,583 (SEQ ID NO:63). A nucleotide and amino acid sequence of a wild type human IMPDH type I is disclosed in Natsumeda, Y., et al. 1990 J. Biol. Chem. 265:5292-5295 (SEQ ID NO:65). Other wild type human IMPDH type I sequences have also been disclosed (Gu et al., 1997 J. Biol. Chem. 272:4458-4466 (SEQ ID NO:62); and Dayton et al. 1994 J. Immunol. 152:984 (SEQ ID NO:64); Zimmermann et al., J. Biol. Chem. 270:6808-6814 (1995); and Glesne et al. Biochem. And Biophys. Research Communications, 537-544 (1994)). Additionally, both IMPDH type I and type II form active tetramers in solution, with subunit molecular weights of 56 kDa (Yamada, Y., et al., 1988 Biochemistry 27:2737-2745).

IMPDH is important for proliferating B and T lymphocytes, as they depend upon the de novo pathway rather than the salvage pathway, to produce nucleotide levels that are required to initiate a proliferative response to mitogens or antigens (Allison, A. C., et. al., 1975 Lancet II, 1179; Allison, A. C., et. al., 1977 Ciba Found. Symp. 48:207). IMPDH also plays a role in the proliferation of smooth muscle cells (Gregory, C. R., et al., 1995 Transplantation 59:655-61). Additionally, IMPDH plays a role in viral replication in certain viral cell lines (Carr, S. F., et al., 1993 supra). Thus, IMPDH is important for diseases involving proliferation of B and T lymphocytes or viral diseases.

Mycophenolic acid (MPA), is a potent uncompetitive, reversible inhibitor of human type I and II IMPDH (Franklin, T. J., and Cook, J. M., 1969 Biochem. J. 113:515-524). MPA binds to IMPDH, after NADH is released, but before XMP is produced (Hedstrom, L. and Wang, C. C. 1990 Biochemistry 29:849-854; Link, J. 0. and Straub, K. 1996 J. Am. Chem. Soc. 118:2091-2092). The reported K_{I} values for human type I IMPDH vary and range between 11 nM (Hager, P. W., et al., 1995 Biochem. Pharmacol. 49:1323-1329) to 33-37 nM (Carr, S. F., et al., 1993 supra), while the K_{I} values for type II are 6-10 nM (Carr, S. F., et al., 1993 supra; Hager, P. W., et al., 1995 supra).

MPA has been used as an immunosuppressant to block the response of B and T cells to mitogen or antigen (Allison, A. C., et. al., 1993 Ann. N.Y Acad. Sci. 696:63). MPA has also been used in the treatment of kidney transplant rejection and autoimmune diseases (Morris, R. E. 1996 Kidney Intl. 49, Suppl. 53:S-26). However, MPA has undesirable pharmacological properties, such as gastrointestinal toxicity and poor bioavailability (Shaw, L. M., et. al., 1995 Therapeutic Drug Monitoring 17:690-699). Other inhibitors of IMPDH activity have been identified, such as nucleoside analogs including tiazofurin, ribavirin and mizoribine (Hedstrom, L. et al., 1990 supra). However, these compounds are competitive inhibitors of IMPDH and lack specificity.

Previous research results do not indicate whether type I or II human IMPDH is the important therapeutic target. For example, it has been demonstrated that levels of IMPDH type II increase in proliferating lymphatic and leukemic cell lines (Konno, Y., et al., 1991 J. Biol. Chem. 266:506-509; Nagai, M., et al., 1991 Cancer Res. 51:3886-3890; Nagai, M., et al., 1992 Cancer Res. 52.258-261; Collart, F. R., et al., 1992 Cancer Res. 52:5). Other researchers demonstrated that mRNA levels of both isoforms increase in T cells following mitogenic stimulation (Dayton, J. S., et al., 1994 J. Immunol. 152:984-991).

IMPDH is integral to the de novo synthesis of guanine nucleotides, e.g., IMPDH catalyzes inosine-5′-monophosphate (IMP) to xanthosine-5′-monophosphate (XMP) (Jackson R. C. et. al., 1975 supra). Therefore, inhibiting the functional activity of IMPDH halts DNA synthesis (Duan, et al., 1987 Cancer Res. 47:4047-4051). There remains a need for potent IMPDH inhibitor molecules of both wild type and modified IMPDH with improved pharmacological properties, in order to inhibit diseases associated with abnormal levels of IMPDH.

Thus, research efforts are currently concentrating on identifying agents that selectively inhibit the activity of wild type IMPDH. Such inhibiting agents are potentially therapeutic inhibitors for use as immunosuppressants, anti-cancer agents, anti-vascular hyperproliferative agents and anti-viral agents. Additionally, the IMPDH inhibitors may be used in the treatment of transplant rejection, and autoimmune diseases, including rheumatoid arthritis, multiple sclerosis, juvenile diabetes, asthma, and inflammatory bowel disease. Additionally, these inhibitors may be useful in the treatment of diseases such as cancer and vascular diseases including restenosis, and viral replication diseases including retroviral diseases and herpes.

One method of discovering agents that inhibit a target protein includes structure-based methods, which involve analysis of the X-ray crystal structures of the target protein complexed with a known inhibitor. The X-ray crystal structure of a multimer of wild-type Chinese hamster IMPDH type II complexed with MPA and IMP has been resolved at the 2.6 Angstrom level (Sintchak, M. D., et al., 1996 supra; U.S. Pat. No. 6,128,582). However, this level of resolution does not provide enough details of the interactions between IMPDH and MPA. There still exists a need to obtain X-ray crystal structures of IMPDH complexed with an inhibitor at a level of resolution that will provide these details. The present invention provides human IMPDH polypeptides which are modified to include a substitute oligo-peptide that replaces the subdomain region. This modified IMPDH polypeptide is shorter than wild-type IMPDH, exhibits functional activity, binds to MPA, and the crystal structure can be resolved at a higher level or finer resolution.

### SUMMARY OF THE INVENTION

The present invention provides, novel isolated modified IMPDH polypeptides and the nucleic acid molecules encoding them. The polypeptides of the invention each include a short substitute oligo-peptide, which replaces the subdomain of the wild-type IMPDH polypeptide, thereby reducing the overall length of the modified IMPDH polypeptide and permitting better resolution of the X-ray crystal structure of the modified IMPDH multimers complexed with an inhibitor. The substitute oligo-peptides have selected lengths and sequences which permit the folded modified IMPDH polypeptide to bind to inhibitors of IMPDH and/or retain the functional activity of wild-type IMPDH.

The invention further provides recombinant vectors and host-vector systems containing DNA encoding the modified IMPDH, and methods for the production of the modified IMPDH polypeptides. The invention also provides antibodies reactive with the modified IMPDH polypeptides.

The modified IMPDH polypeptides are useful for drug discovery methods, such as structure-based drug design. The modified IMPDH polypeptides of the invention are also useful for therapeutic, diagnostic and prognostic procedures for the detection and/or quantification of the modified IMPDH polypeptides, as well as for the detection and/or quantification of the corresponding nucleic acids molecules that encode any IMPDH polypeptides.

### BRIEF DESCRIPTION OF THE FIGURES

FIG. 1 depicts the amino acid sequence of a type I, wild-type, human IMPDH polypeptide (SEQ ID NO:48).

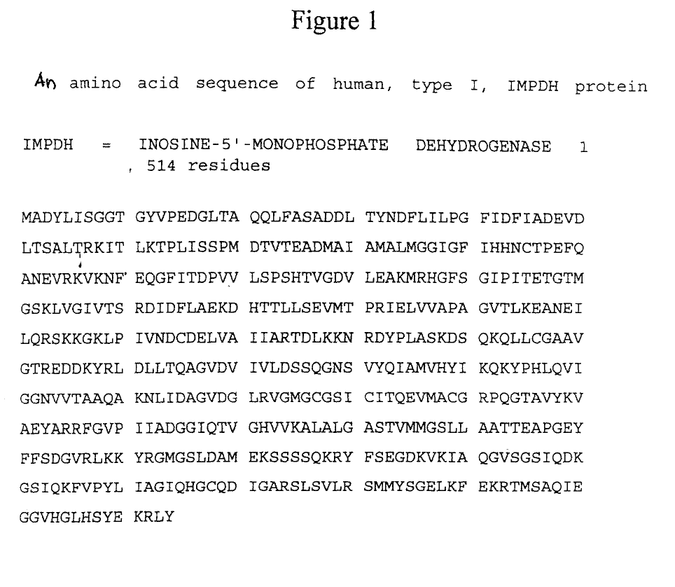

FIG. 2 depicts the amino acid sequence of a type II, wild-type, human IMPDH polypeptide (SEQ ID NO:49).

FIG. 3 is a schematic representation of a modified IMPDH polypeptide including a substitute oligo-peptide that replaces the subdomain region of the wild type IMPDH polypeptide.

FIG. 4 depicts the amino acid sequence of type II, IMPDH-DKT polypeptide of the invention (SEQ ID NO:20).

FIG. 5 depicts the nucleotide sequence of type II, IMPDH-DKT cDNA of the invention (SEQ ID NO:40).

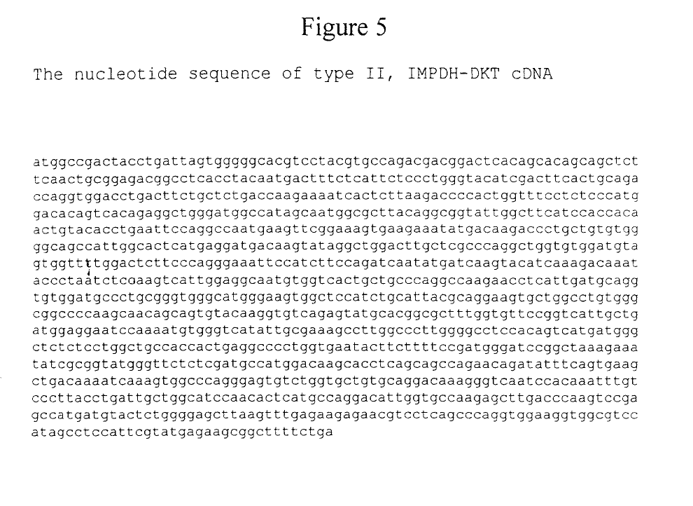

FIG. 6 depicts the amino acid sequence of type II, IMPDH-SPS polypeptide of the invention (SEQ ID NO:22).

FIG. 7 depicts the nucleotide sequence of type II, IMPDH-SPS cDNA of the invention (SEQ ID NO:41).

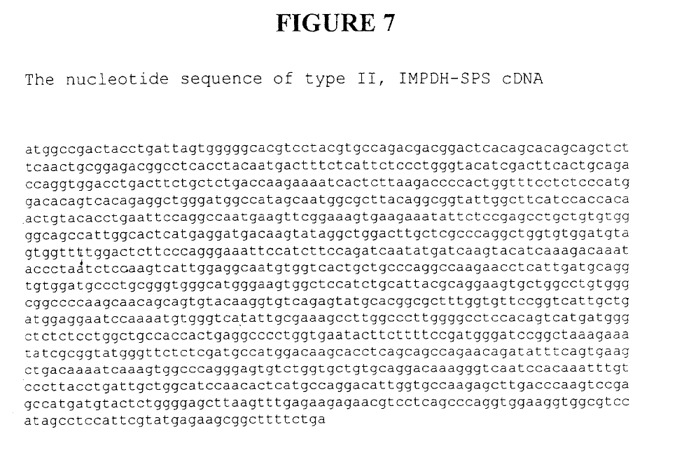

FIG. 8 depicts the amino acid sequence of type II, IMPDH-GSG polypeptide of the invention (SEQ ID NO:29).

FIG. 9 depicts the nucleotide sequence of type II, IMPDH-GSG cDNA of the invention (SEQ ID NO:42).

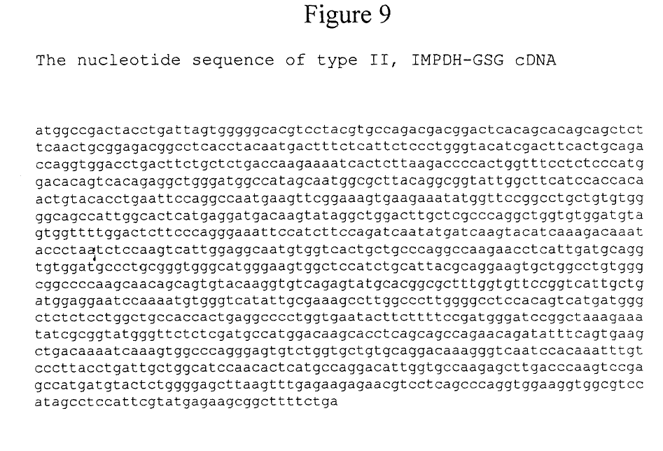

FIG. 10 depicts the amino acid sequence of type II, IMPDH-SPT polypeptide of the invention (SEQ ID NO:27).

FIG. 11 depicts the nucleotide sequence of type II, IMPDH-SPT cDNA of the invention (SEQ ID NO:43).

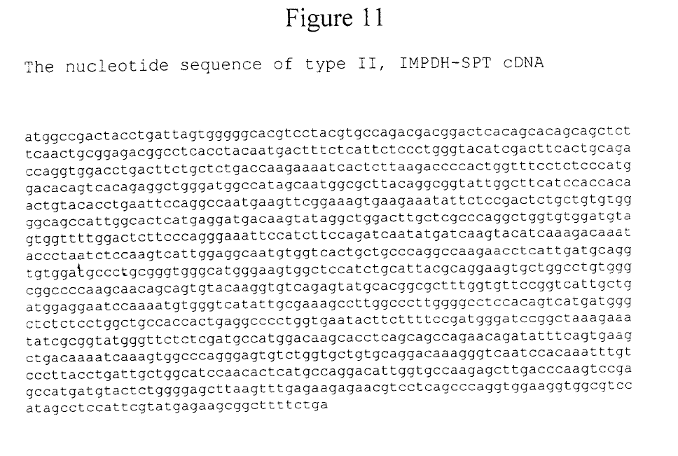

FIG. 12 depicts the nucleotide sequence of type II, IMPDH-SPTQ cDNA of the invention (SEQ ID NO:45).

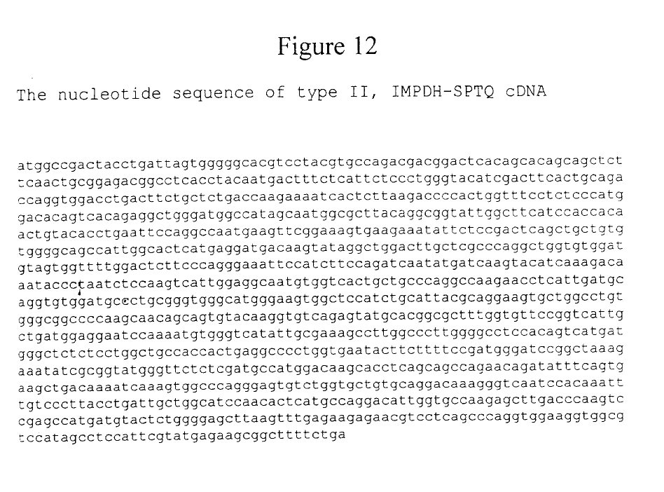

FIG. 13 depicts the amino acid sequence of type II, IMPDH-AGRP polypeptide of the invention (SEQ ID NO:36).

FIG. 14 depicts the nucleotide sequence of type II, IMPDH-AGRP cDNA of the invention (SEQ ID NO:46).

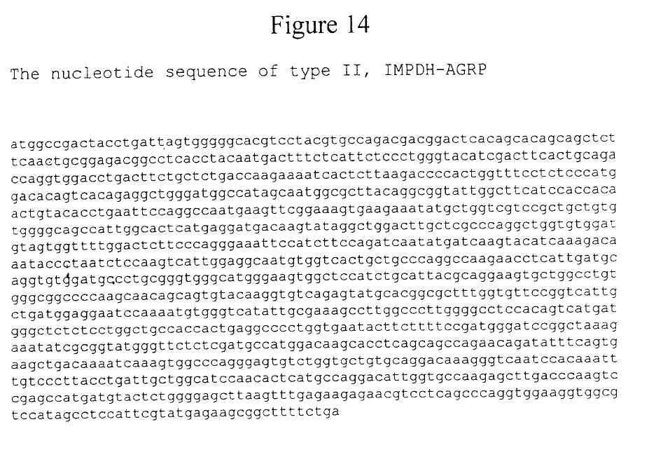

FIG. 15 depicts the amino acid sequence of type II, IMPDH-NSPL polypeptide of the invention (SEQ ID NO:38).

FIG. 16 depicts the nucleotide sequence of type II, IMPDH-NSPL cDNA of the invention (SEQ ID NO:47).

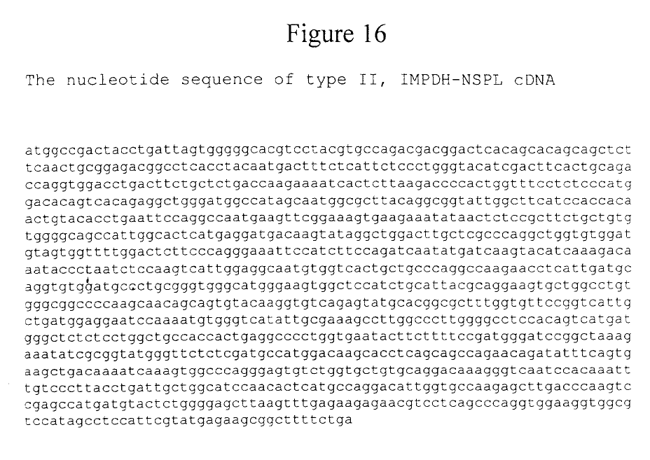

FIG. 17 depicts the amino acid sequence of type I, IMPDH-DKT polypeptide of the invention (SEQ ID NO:30).

FIG. 18 is a ribbon diagram depicting a model of the folded wild-type human, type II IMPDH protein used to design the substitute tri- and tetra-peptides, as described in Example 1, infra.

FIG. 19 is a bar graph illustrating detection of NADH production from protein multimers comprising modified IMPDH polypeptides, as described in Example 2, infra.

FIG. 20 is a graph demonstrating that MPA inhibits the enzymatic activity of modified IMPDH polypeptides, as described in Example 3, infra.

FIG. 21 is a Coomassie-stained Tris-Glycine gel showing the isolated modified IMPDH-DKT polypeptide, as described in Example 4, infra.

FIGS. 22A-C depict a HPLC-EMS trace of the isolated modified IMPDH-DKT polypeptide, as described in Example 4, infra. A) chromatogram of IMPDH-DKT polypeptide showing total ion current versus elution time; B) integrated mass spectrum showing normalized intensity versus mass/charge for the protein eluting at 11.03 min; C) reconstructed spectrum of data in FIG. 13B for protein eluting at 11.03 Min.

FIG. 23 illustrates a Gel Permeation chromatograph of the isolated modified IMPDH-DKT polypeptide, as described in Example 4, infra.

FIG. 24 depicts the nucleotide sequence of type I, IMPDH-DKT cDNA of the invention (SEQ ID NO:44).

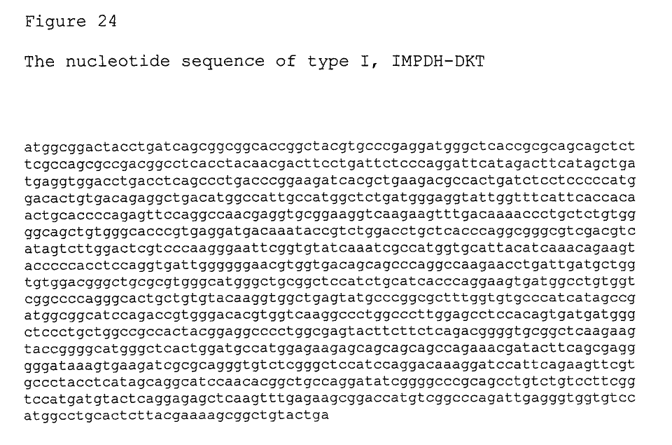

### DETAILED DESCRIPTION OF THE INVENTION

Definitions

All scientific and technical terms used in this application have meanings commonly used in the art unless otherwise specified. As used in this application, the following words or phrases have the meanings specified.

As used herein, the term “holo-enzyme” refers to a complete, functional enzyme comprising multiple components, such as polypeptide subunits and cofactors, which are associated with each other to make up the holo-enzyme. The polypeptide subunits may be associated with each other by covalent or non-covalent interactions.

As described herein, the term “IMPDH holo-enzyme” refers to a complete enzyme having the biological activity or function of catalyzing the nicotinamide adenine dinucleotide (NAD)-dependent oxidation of inosine-5′-monophosphate (IMP), which is the committed step in de novo synthesis of guanine nucleotides. Typically, the naturally-occurring IMPDH holo-enzyme is found as a tetramer molecule comprising four monomer subunits of IMPDH polypeptides each bound to a potassium ion. Additionally, the catalytic activity of wild-type human IMPDH holo-enzyme is inhibited by mycophenolic acid (MPA).

As described herein, the term “IMPDH multimer” refers to at least two monomer subunits of IMPDH polypeptides associated with each other to form an IMPDH multimer molecule. The polypeptide subunits may be associated with each other by covalent or non-covalent interactions. The IMPDH multimer may or may not include potassium ions. The IMPDH multimer may or may not exhibit functional activity of naturally-occurring IMPDH holo-enzyme. The IMPDH multimer may comprise two, three, four, or up to eight monomer subunits of modified IMPDH polypeptides. Further, the multimer may comprise monomer subunits that are identical or different isoforms, such as modified type I or type II IMPDH polypeptides.

As used herein, the term “wild type IMPDH polypeptide” refers to a polypeptide that includes an N-terminal catalytic domain, an internal non-catalytic domain, and a C-terminal catalytic domain. As discussed herein, the wild type IMPDH polypeptide may vary in its amino acid sequence. Examples of wild type IMPDH polypeptides are provided in SEQ ID NOS:48, 49, and 62-65. The wild type IMPDH polypeptide folds into a structure, such that the N-terminal and C-terminal domains form a catalytic site The non-catalytic domain is also known as the flanking domain or the subdomain. The wild type IMPDH polypeptide is a monomer polypeptide subunit of the wild type IMPDH holo-enzyme or of the wild type IMPDH multimer.

As used herein, the term “modified IMPDH polypeptide” refers to an IMPDH polypeptide having the IMPDH subdomain (e.g., internal non-catalytic domain) of the wild type IMPDH polypeptide substituted with an oligo-peptide, designated herein as the “substitute peptide”.

As used herein, the term “oligo-peptide domain” refers to the region or portion of the modified IMPDH polypeptide where the oligo-peptide has replaced the wild type IMPDH subdomain (e.g., the internal non-catalytic domain).

As used herein, the term “substitute peptide” or “substitute oligo-peptide” or “substitute tetra-peptide” or “substitute tri-peptide” refers to a peptide fragment, having a selected length and sequence, that substitutes for the subdomain region of the wild type IMPDH polypeptide. The peptide fragment is smaller than the subdomain region (e.g., it has fewer than 133 amino acids). In preferred embodiments, the peptide fragment is a tri-peptide or a tetra-peptide.

As used herein, a first nucleotide or polypeptide sequence is said to have sequence “identity” to a second nucleotide or polypeptide sequence when a comparison of the first and the second sequences shows that they are exactly alike.

As used herein, a first nucleotide sequence is said to be “similar” to a second reference sequence when a comparison of the two sequences shows that they have a low level of sequence differences. For example, two sequences are considered to be similar to each other when the percentage of nucleotides that differ between the two sequences may be between about 60% to 99.99%.

The term “complementary” as used herein refers to the capacity of purine and pyrimidine nucleotides to associate through hydrogen bonding to form double-stranded nucleic acid molecules. The following base pairs are related by complementarity: guanine and cytosine; adenine and thymine; and adenine and uracil. The term “complementary” applies to all base pairs comprising two single-stranded nucleic acid molecules.

As used herein, a nucleic acid molecule encoding a polypeptide of interest is said to be “isolated” when the nucleic acid molecule is substantially separated from contaminant nucleic acid molecules that encode polypeptides other than the polypeptide of interest.

As used herein, a polypeptide of interest is said to be “isolated” when the polypeptide of interest is substantially separated from other “contaminant” polypeptides. Additionally, an “isolated” nucleic acid molecule or polypeptide refers to any DNA, RNA, or polypeptide sequence, however constructed or synthesized.

As used herein, “naturally occurring” refers to a polypeptide that is found in nature.

“Substantially purified” as used herein means a specific isolated nucleic acid or polypeptide, or fragment thereof, in which substantially all contaminants (i.e. substances that differ from the specific molecule) have been separated from said nucleic acid or protein.

The single-letter codes for amino acid residues include the following: A alanine, R arginine, N=asparagine, D=aspartic acid, C cysteine, Q Glutamine, E=Glutamic acid, G=glycine, H=histidine, I=isoleucine, L=leucine, K=lysine, M=methionine, F=phenylalanine, P=proline, S=serine, T=threonine, W=tryptophan, Y=tyrosine, V=valine.

In order that the invention herein described may be more fully understood, the following description is set forth.

A. Molecules of the Invention

In its various aspects, as described in detail below, the present invention provides isolated, modified IMPDH polypeptides, nucleic acid molecules, recombinant DNA molecules, transformed host cells, generation methods, assays, immunotherapeutic methods, transgenic animals, inhibitors of modified IMPDH polypeptides (e.g., antibodies), immunological and nucleic acid-based assays, and compositions.

1. Modified IMPDH Polypeptides and Multimeric Molecules Comprising Modified IMPDH Polypeptides

a.) Isolated Modified IMPDH Polypeptides

Modified IMPDH polypeptides of the present invention, and fragments thereof, may be embodied in many forms, preferably in isolated form. Polypeptides of the invention may be isolated as naturally-synthesized polypeptides or from any source whether natural, synthetic, semi-synthetic, or recombinant. Accordingly, the modified IMPDH polypeptides may be isolated as a naturally-synthesized protein from any species, particularly mammalian, including bovine, ovine, porcine, murine, equine, and preferably human. Alternatively, the modified IMPDH polypeptides may be isolated as a recombinant polypeptide that is expressed in prokaryote or eukaryote host cells, or isolated as a synthetic polypeptide.

A skilled artisan can readily employ standard isolation methods to obtain an isolated, modified IMPDH protein, prepared according to the methods of the invention. The nature and degree of isolation will depend on the source and the intended use of the isolated protein. For example, modified IMPDH polypeptides can be isolated from bacterial host cells using the methods used to isolate naturally-occurring bacterial IMPDH proteins as described in Gilbert et al., (1979) Biochemical J. 183:481-494 and Krishnaiah (1975) Arch. Biochem. Biophys. 170:567-575. Alternative methods can be used to isolate modified IMPDH from eukaryotic cells, such as plant cells (Atkins, et al., (1985) Arch. Biochem. Biophys. 236:807-814) or Chinese hamster cells (Collart, et al., (1987) Mol. Cell. Biol. 7:3328-3331) or Yoshida sarcoma ascites cells (Okada, et al., (1983) J. Biochem. 27:2193-2196) or rat hepatoma cells (Ikegami, et al., (1987) Life Sci. 40:2277-2282).

The methods for generating modified IMPDH polypeptides are described in detail below.

b.) Purified Modified IMPDH Polypeptides

Modified IMPDH polypeptides of the invention, and fragments thereof, may be isolated in purified form. The modified IMPDH polypeptides can be purified by methods well known in the art, including, affinity chromatography using IMP or anti-IMPDH antibodies (Marchak, D. R., et al., 1996 in: Strategies for Protein Purification and Characterization, Cold Spring Harbor Press, Plainview, N.Y.). The nature and degree of isolation and purification will depend on the intended use. For example, purified, modified IMPDH polypeptides will be substantially free of other proteins or molecules that impair the binding of ligands or antibodies to the modified IMPDH polypeptides.

c.) The Crystallized Modified IMPDH Polypeptides

The modified IMPDH polypeptides of the invention, and fragments thereof, can be isolated in crystal form, using methods well known in the art (Fleming, M. A., et al., 1996 Biochemistry 35, 6990-6997).

d.) The Modified IMPDH Polypeptides Have Substituted Subdomains

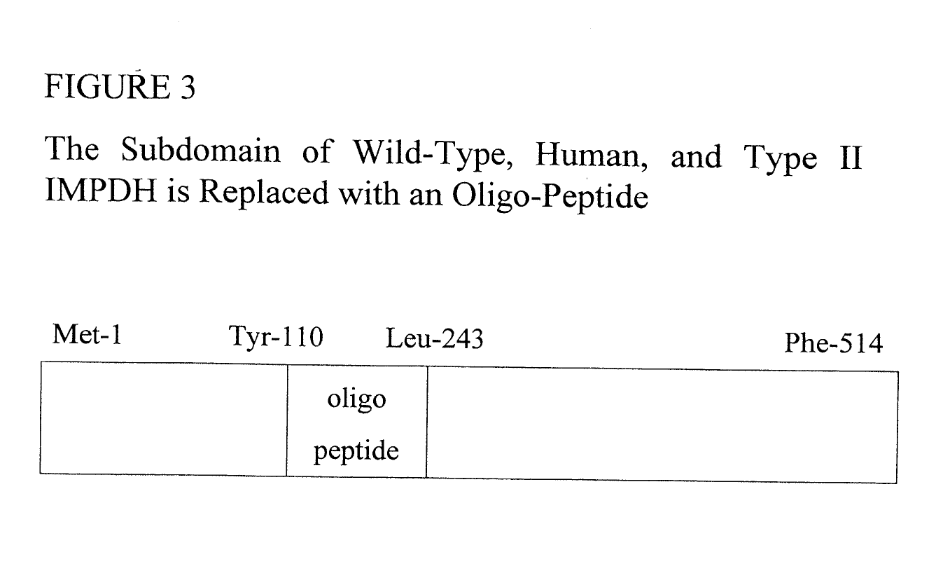

The present invention provides modified IMPDH polypeptides each including: the N-terminal catalytic core domain of wild type IMPDH; an internal substitute oligo-peptide domain that replaces the wild type IMPDH subdomain region, or replaces a region within the subdomain region; and the C-terminal catalytic core domain of wild type IMPDH. The N- and C-terminal catalytic core domains of the modified IMPDH polypeptide may include type I or type II, wild-type sequences from various sources, such as GenBank, EC 1.1.1.205, or International Publication Number WO94/24264. Additionally, the N- or C-terminal sequences may include sequences identical to that described in Collart, F. R. and Hubermann, E. 1988 J. Biol. Chem. 263:15769-15772 or Natsumeda, Y., et al. 1990 J. Biol. Chem. 265:5292-5295, or as indicated in SEQ ID NOS: 48, 49, and 62-65, herein. The oligo-peptide that replaces the wild type subdomain region (FIG. 3) will be referred to generally as the “substitute oligo-peptide”, and more particularly as the “substitute tri-peptide” or “substitute tetra-peptide”, depending on the length of the oligo-peptide.

The purpose of the substitute oligo-peptide is to reduce the overall length of the modified IMPDH polypeptide, so as to obtain better resolution of the X-ray crystal structure of the modified IMPDH multimers complexed with an inhibitor, such as MPA. Additionally, the sequence and length of the substitute oligo-peptide are selected to permit the folded IMPDH polypeptides and multimers to bind to inhibitors of IMPDH and/or retain the functional activity of wild-type IMPDH (e.g., produce NADH).

The present invention also provides fragments of the modified IMPDH polypeptides, where the fragments include the substitute oligo-peptide that replaces the subdomain region of wild-type type I or II IMPDH.

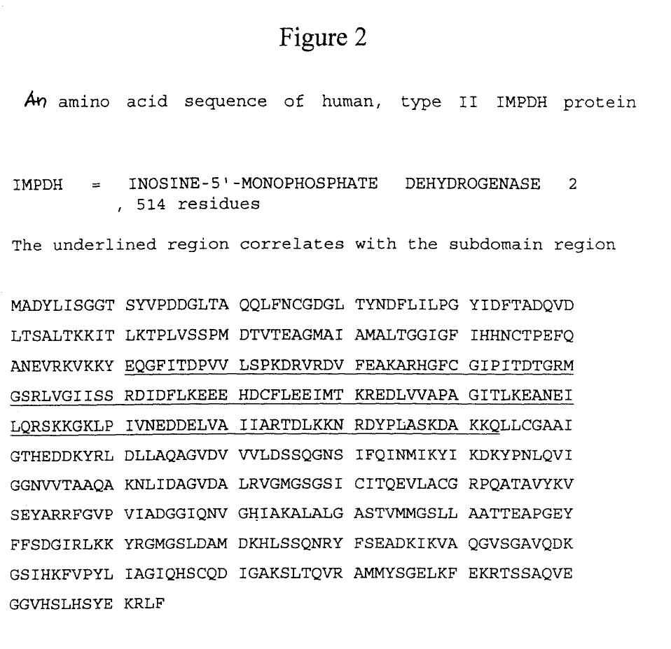

The previously determined amino acid sequences of wild-type, human, type I (Natsumeda, Y., et al. 1990 J. Biol. Chem. 265:5292-5295; SEQ ID NO:65; Gu et al., 1997 J. Biol. Chem. 272:4458-4466 (SEQ ID NO:62); and Dayton et al. 1994 J. Immunol. 152:984 (SEQ ID NO:64)) or a type II (FIG. 2; SEQ ID NO.:49) (Natsumeda, Y., et al. 1990 J. Biol. Chem. 265:5292-5295; Collart, F. R. and Hubermann, E. 1988 J. Biol. Chem. 263:15769-15772; and U.S. Pat. No. 5,665,583 (SEQ ID NO:63); Zimmermann et al., J. Biol. Chem. 270:6808-6814 (1995); and Glesne et al. Biochem. And Biophys. Research Communications, 537-544 (1994)) IMPDH polypeptide may be used to locate the wild type subdomain regions. Or, amino acid sequences as shown in SEQ ID NOS:48, 49 or 62-65 may be used to locate the subdomain regions. Alternatively, the amino acid sequence of wild type Chinese hamster IMPDH (Collart, F. R. and Hubermann, E. 1988 supra), or the wild type IMPDH protein from prokaryotes and other eukaryotes (Natsumeda, Y. and Carr, S. F., 1993 supra) may also be used to locate the wild type subdomain regions.

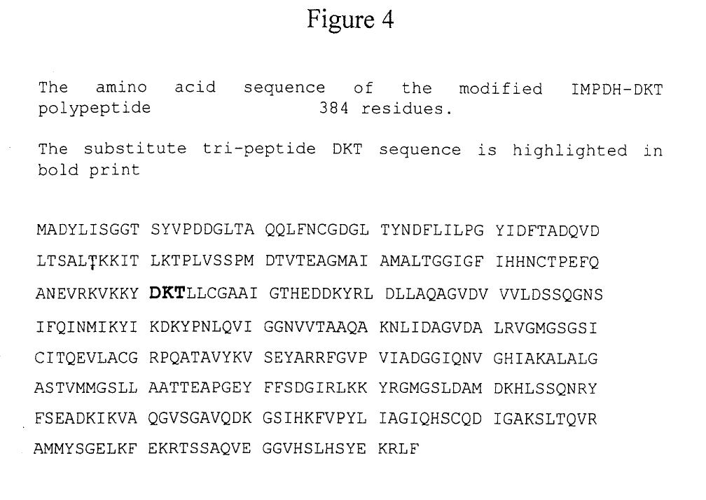

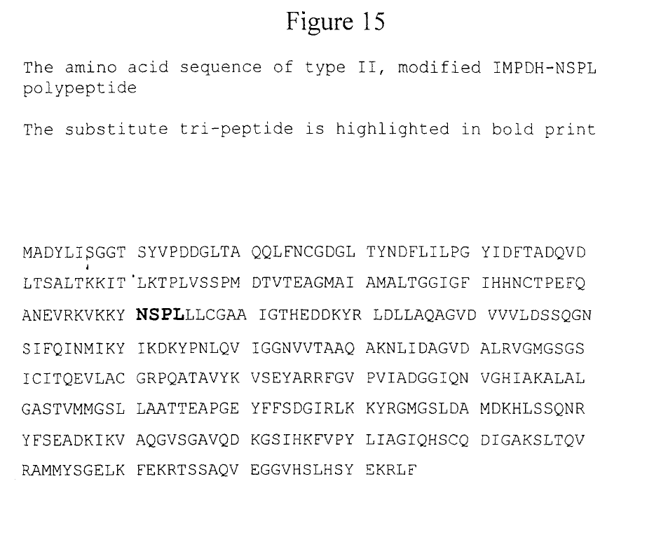

For example, the modified, human, wild type type II IMPDH polypeptides include, but are not limited to: (1) the N-terminal catalytic core domain encompassing residues 1-110; (2) the internal non-catalytic subdomain encompassing residues Glu111-Gln243 substituted with the oligo-peptide; and (3) the C-terminal catalytic core domain encompassing residues 244-514. Thus, the substitute oligo-peptide links the two catalytic core domains (1 and 3) and reduces the overall length of the modified IMPDH polypeptide compared to wild-type type II IMPDH (FIGS. 4, 6, 8, 10, 13, 15, or 17). The length of the modified IMPDH polypeptide will depend on the length of the oligo-peptide. For example, if the oligo-peptide is a tri-peptide, the modified IMPDH polypeptide will be 384 amino acid residues in length. The preferred sequence and length of the substitute oligo-peptides are selected, such that the modified IMPDH polypeptides fold to form a functionally catalytic core. The folded, modified IMPDH polypeptides bind inhibitors of IMPDH activity (e.g., produce NADH).

e.) The Size of the Substitute Oligo-peptides

The present invention provides modified IMPDH polypeptides including substitute oligo-peptides that are between about 3 to 10 amino acid residues in length. A preferred embodiment provides modified IMPDH polypeptides comprising substitute oligo-peptides that are tri-peptides. Additionally, a preferred embodiment provides modified IMPDH polypeptides comprising substitute oligo-peptides that are tetra-peptides.

f.) The Sequence of the Substitute Oligo-peptides

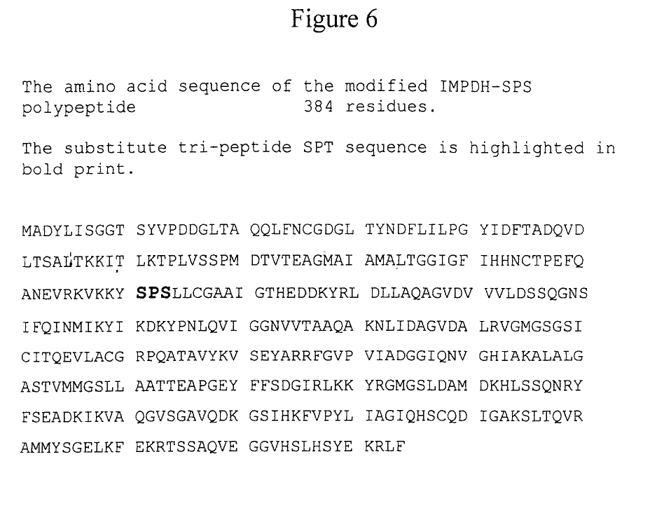

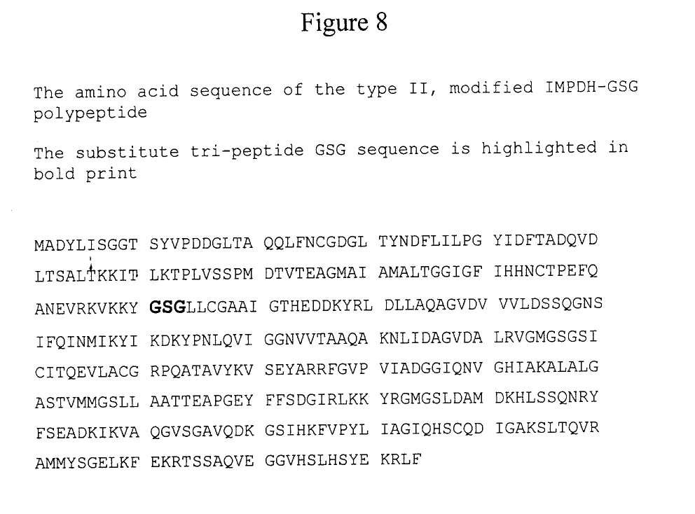

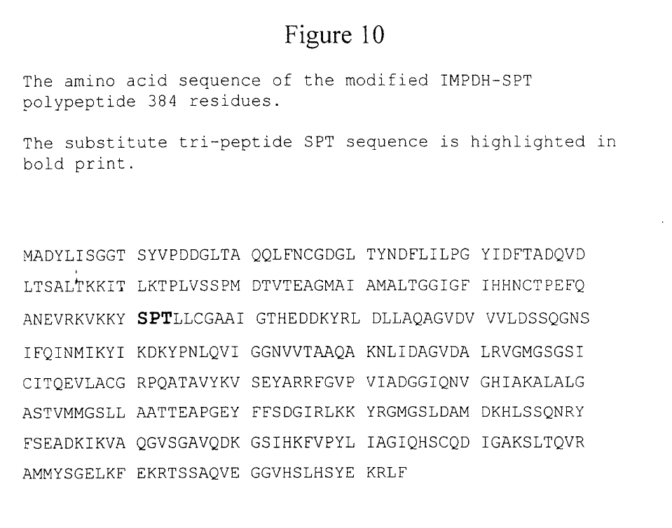

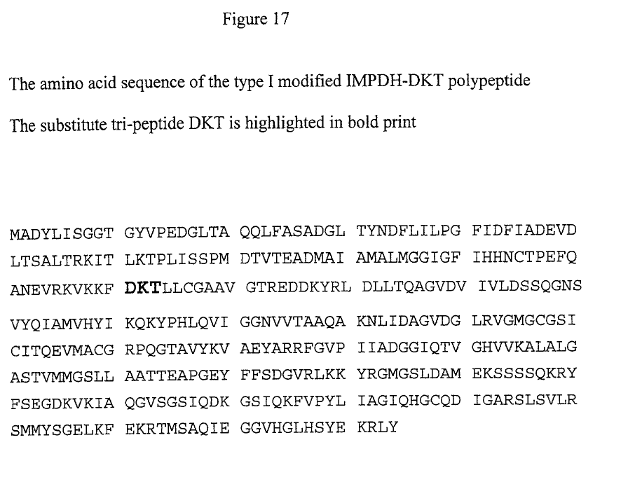

The present invention also provides modified IMPDH polypeptides (SEQ ID NO.:20-30) comprising substitute oligo-peptides having amino acid sequence identity to any one of the substitute tri-peptide sequences, as described in SEQ ID NOS.: 1-10. For example, the modified IMPDH polypeptide may comprise a substitute tri-peptide having any one of the sequences (in single amino acid codes): DKT, TPI, SPS, SAH, KPI, IVD, ALF, SPT, GGY or GSG (SEQ ID NO.:1-10). The preferred embodiment provides modified IMPDH polypeptides each comprising a different substitute tri-peptide, such as the type I IMPDH-DKT (FIG. 17, SEQ ID NO.: 30) or type II IMPDH-DKT (FIG. 4, SEQ ID NO.:20), or type II IMPDH-SPS (FIG. 6, SEQ ID NO.22), IMPDH-SPT (FIG. 10, SEQ ID NO.:27), and IMPDH-GSG (FIG. 8, SEQ ID NO.:29).

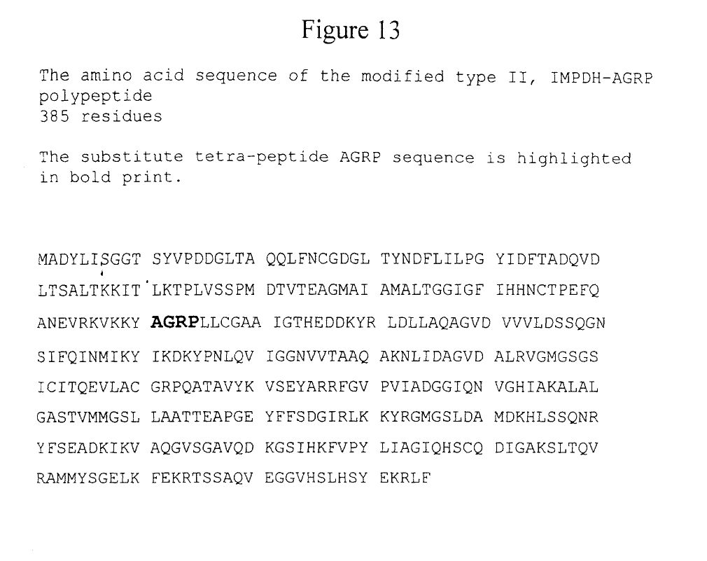

The present invention provides additional modified IMPDH polypeptides (SEQ ID NO.:31-39) comprising substitute oligo-peptides having amino acid sequence identity to any one of the substitute tetra-peptide sequences, as described in SEQ ID NOS.: 11-19. For example, the modified IMPDH polypeptide may comprise a substitute tetra-peptide having any one of the sequences: GSSW, QPQS, NIIP, SPTQ, TRYT, AGRP, NGQY, NSPL, or YGTW (SEQ ID NO.: 11-19). The preferred embodiment provides a modified IMPDH polypeptide comprising a substitute tetra-peptide, such as the IMPDH-AGRP (FIG. 13, SEQ ID NO.: 36).

g.) The Variant Sequences of the Substitute Oligo-peptides

The present invention also provides modified IMPDH polypeptides comprising substitute oligo-peptide regions having sequence variations of the substitute tri- or tetra-peptide regions described herein. For example, variants of the modified IMPDH polypeptides may differ, by one or more amino acid substitutions, from the substitute tri- or tetra-peptide sequences described in SEQ ID NOS.: 1-10 or SEQ ID NOS.: 11-19, respectively. The amino acid substitutions may be conservative changes, where a substituted amino acid has similar structural or chemical properties, e.g., replacement of leucine with isoleucine. Variants may also have nonconservative changes, e.g., replacement of a glycine with a tryptophan. Guidance in determining which and how many amino acid residues may be varied in the substitute oligo-peptide regions, may be found in the distance spanned by the subdomain in a folded wild-type mammalian IMPDH multimer, where the spanned distance is derived by either prediction (e.g., based on amino acid sequence) and/or experiment (e.g., based on X-ray crystallography). Amino acids are preferably varied so that the spanned distance is identical or nearly identical to the distance spanned by the wild type subdomain.

As is well known in the art, a polynucleotide sequence can encode amino acid substitutions without altering either the conformation or the function of the polypeptide. With respect to the invention herein, amino acid substitutions can occur either in the substitute oligopeptide, or in any portion of the polypeptide (e.g., the N-terminal catalytic domain, the subdomain, or in the C-terminal catalytic domain). Conservative amino acid changes include, but are not limited to, substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A), or glycine (G) and serine (S) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

In additional embodiments of the invention, the amino acid substitutions can be “nonconservative”. Examples include, but are not limited to, aspartic acid (D) being replaced with glycine (G); asparagine (N) being replaced with lysine (K); or alanine (A) being replaced with arginine (R).

In addition, the present invention provides modified IMPDH polypeptides comprising substitute oligo-peptide regions (e.g. substitute tripeptides or substitute tetrapeptides), and having sequence variations in the N-terminal catalytic domain and/or in the C-terminal catalytic domain. Preferably, such substitutions do not alter the functional activity of the wild type or modified IMPDH polypeptides. Examples of variations in the N-terminal catalytic domain are indicated in SEQ ID NO:62. In particular, aspartic acid (D) at position 29 (D29; SEQ ID NO:48) can be replaced with glycine (G). In addition, asparagine (N) at position 109 (N109; SEQ ID NO:48) can be replaced with lysine (K). As persons skilled in the art understand, any number of amino acids can be changed alone, or in combination with other amino acids and yet the polypeptides retain their functional activity (e.g., the IMPDH polypeptides with amino acid substitutions retain the ability to regulate NADH production, as described herein). Thus, any molecule that functions similarly to the wild type or modified IMPDH can be used to practice the invention described herein.

Examples of different nucleotide or amino acid sequences of IMPDH can be found in Natsumeda et al. J. Biol. Chem. 265:5292-5295 (1990); Collart, F. R. and Hubermann, E. 1988 J. Biol. Chem. 263:15769-15772; and U.S. Pat. No. 5,665,583; Gu et al. J. Biol. Chem. 272:4458-4466 (1997); Dayton et al. J. Immunol. 152:984 (1994); Zimmermann et al., J. Biol. Chem. 270:6808-6814 (1995); and Glesne et al. Biochem. And Biophys. Research Communications, 537-544 (1994). These amino acid sequences are also indicated herein in SEQ ID NOS:62-65.

Particular examples of variants of the IMPDH-DKT polypeptides of the invention having conservative amino acid substitutions in the substitute tri-peptide region (e.g., DKT) include, but are not limited to (in single-letter code): GKT, DRT, DKG, or GRS. One skilled in the art can readily contemplate other variants of the substitute oligo-peptide sequences.

h.) Amino Acid Analogs or Polypeptides Which are Altered

The present invention further provides modified IMPDH polypeptides comprising amino acid analogs. The amino acid analogs may be chemically synthesized, and include dextro or levo forms, or peptidomimetics. The present invention also provides polypeptides of the invention which are altered, for example, by post-translational pathways or by chemical synthesis, including N- or O-glycosylated amino acid residues. The N-terminal end of the polypeptides may be altered to include acylated or alkylated residues. The C-terminal end of the polypeptides may be altered to include esterified or amidated residues. The non-terminal amino acid residues may be altered, including but not limited to, alterations of the amino acids, arginine, aspartic acid, asparagine, proline, glutamic acid, lysine, serine, threonine, tyrosine, histidine, and cysteine.

i.) The Distance Spanned by the Substitute Oligo-peptides

The present invention provides modified IMPDH polypeptides comprising a substitute oligo-peptide that spans the distance of the subdomain in a folded wild-type mammalian IMPDH polypeptide, or an IMPDH multimer comprising a plurality of folded modified IMPDH polypeptides which are associated with each other, so that the folded modified IMPDH polypeptides exhibit the functional activity of wild-type IMPDH and/or bind to inhibitors of IMPDH.

The distance that spans the subdomain in a folded wild type IMPDH polypeptide may be predicted from the amino acid sequence of a wild type IMPDH polypeptide and/or obtained experimentally from X-ray crystal structures of the wild type IMPDH polypeptide monomer or the wild type IMPDH multimer or the wild type IMPDH holo-enzyme.

For example, the amino acid sequence of human type I (Natsumeda, Y., et al. 1990 supra) and/or type II (Collart, F. R., and Hubermann, E. 1988 supra; Natsumeda, Y., et al. 1990 supra) IMPDH polypeptides may be used as a basis to predict the distance that spans the subdomain of folded human wild type IMPDH polypeptides. The subdomain of wild-type human type II IMPDH polypeptide, encompassing residues Glu111-Gln243 spans a linear length of 133 amino acid residues (SEQ ID NO.:61).

The x-ray crystal structure of IMPDH may also be used to predict the distance that spans the subdomain in a folded IMPDH polypeptide. For example, the X-ray crystal structure of wild-type Chinese hamster IMPDH holo-enzyme complexed with IMP and MPA has been previously determined and shows that the dimensions of the subdomain is roughly 20×20×40 Angstroms, and the distance that spans the subdomain from Glul 11-CA to Gln243-CA (e.g., CA is the carbon alpha atom for each amino acid residue) is about 5.1 Angstroms (M. D. Sintchak, et al., 1996 Cell 85:921-930). The X-ray crystal structure for human IMPDH has also been determined; the distance from Glu 11-CA to Gln243-CA is about 5.5 Angstroms (Colby, T. D., et al., 1999 Proc. Natl. Acad. Sci. USA 96:3531). It is postulated that the distance that spans the subdomain of the folded type I IMPDH is approximately 5 Angstroms.

The present invention provides the discovery that the distance that spans the length of the substitute tri-peptide in the IMPDH-DKT multimer of the invention bound to MPA is about 5 Angstroms, as determined from X-ray crystal data.

One embodiment of the invention provides modified IMPDH polypeptides each comprising a substitute oligo-peptide that spans the distance of between about 4.8 to 6.0 Angstroms, which is the range of the distance of the subdomain region in a folded wild-type mammalian IMPDH polypeptide monomer or a folded wild-type mammalian IMPDH multimer. A more preferred embodiment provides modified IMPDH polypeptides each comprising a substitute oligo-peptide that spans the distance of between about 5.0 and 5.2 Angstroms.

j.) Multimer Forms From the Modified IMPDH Polypeptides

The present invention provides multimeric IMPDH molecules, herein designated modified IMPDH multimers, comprising a plurality of modified type I and/or type II human IMPDH polypeptides associated with each other. The modified IMPDH multimers may or may not include other components, such as potassium ions. Accordingly, a modified homo-multimeric IMPDH molecule may comprise a plurality of type I or type II modified IMPDH polypeptides. Similarly, a modified hetero-multimeric IMPDH molecule may comprise a combination of type I and type II modified IMPDH polypeptides.

For example, modified homo-meric IMPDH molecules each including a plurality of a particular modified type II human IMPDH polypeptides were isolated, including the IMPDH-DKT, -SPS, -SPT, and -AGRT multimers.

The modified IMPDH multimers may include between two to eight modified IMPDH polypeptides (Carr, S. F., et al., 1993 supra). One embodiment of the invention provides a modified IMPDH multimer having two modified IMPDH polypeptides (e.g., dimer). A more preferred embodiment includes a modified IMPDH multimer having four modified IMPDH polypeptides (e.g., tetramer), and a most preferred modified IMPDH multimer having eight modified IMPDH polypeptides (e.g., octomer).

The modified IMPDH polypeptides may form the multimeric molecules in an appropriate buffered solution, such as, for example a solution including 25 mM Tris, pH 8.2; 300 mM KCl; 10% glycerol; 1 mM EDTA; and 2 mM DTT (Brandon C. & Tooze J. 1991 in: Introduction to Protein Structure Garland Publishing Inc., London).

The formation of tetramers and octamers in solution, can be determined by various methods, including static and dynamic light scattering (Freifelder, D. 1982 in: Physical Biochemistry: Applications to Biochemistry and Molecular Biology, W. H. Freeman & Co., San Francisco, Calif.), and analytical ultracentrifugation (Deutscher, M.P. 1990 in Guide to Protein Purification: Methods in Enzymology, Academic Press, Inc., San Diego, Calif.). Alternative methods include native SDS/PAGE gel electrophoresis and gel permeation chromatography (ed. Freifelder, D. 1982 in: Physical Biochemistry; Applications to Biochemistry and Molecular Biology, W. H. Freeman & Co., San Francisco, Calif.; ed. Oliver, R. W. A. 1989 in: HPLC of Macromolecules. a Practical Approach IRL Press, Oxford University).

For example, the IMPDH-DKT, -SPS, -SPT, and -AGRP multimers appear to be in dynamic equilibrium between the dimer and tetramer multimer forms, as determined by analytical gel permeation chromatography (GPC) (ed. Oliver, R. W. A. 1989 in: HPLC of Macromolecules: a Practical Approach IRL Press, Oxford University).

k.) The Modified IMPDH Multimers Exhibit Functional Activity

The present invention provides modified IMPDH multimers that exhibit the functional activity of the wild-type IMPDH holo-enzyme. For example, the wild-type mammalian IMPDH holo-enzyme catalyzes the conversion of NAD to NADH in the presence of IMP.

Accordingly, the multimeric molecules of the invention catalyze the production of NADH in the presence of IMP. One skilled in the art can readily assay modified IMPDH multimers for the production of NADH, using the in vitro methods described herein, or as described by S. F. Carr, et al. (1993) J. Biol. Chem. 268:27286-27290 and also B. Xiang, et al. (1996) J. Biol. Chem. 271:1435-1440.

For example, the amount of NADH produced by the modified IMPDH multimers and the wild-type human type II IMPDH holo-enzyme may be compared by spectrophotometric measurements at 340 nm (±=6220 M⁻¹ cm⁻¹) at 37° C. (Carr, S. F., et al., 1993 supra).

Alternatively, the functional activity of the multimeric molecules of the invention can be analyzed by measuring the production of XMP and NADH from IMP and NAD, using HPLC analysis and spectrophotometric assays (Montero, C. et al., 1995 Clinica Chemica Acta 238:169-178).

1.) The Inhibitory Effect of MPA and/or Other Compounds on Modified IMPDH Polypeptides

The present invention provides modified IMPDH multimers, having the functional activity of the wild-type IMPDH holo-enzyme, that are inhibited by compounds known to inhibit the activity of wild-type mammalian IMPDH holo-enzyme. For example, MPA is a compound that is a non-competitive inhibitor of IMPDH holo-enzyme activity, which will inhibit the conversion of NAD to NADH (T. J. Franklin and J. M. Cook 1969 Biochem. J. 113:515-524). Alternative compounds include rapamycin and nucleoside analogs that are competitive inhibitors, such as tiazofurin, ribavirin and mizoribine (Hedstrom, L., et. al., 1990 Biochemistry 29:849-854; Cooney, D., et al., 1982, Biochem. Pharm. 31: 2133-2136; Smith, C., et al., 1974: Biochem. Pharm., 23: 2727-2735; Koyama, H. & Tsuji, M., 1983, Biochem. Pharm., 32: 3527-3553).

Typically, the method to evaluate the effect of a putative inhibitory compound includes the following steps: the wild-type holo-enzyme and the modified IMPDH multimer are placed in separate reaction vessels each containing the appropriate concentrations of IMP, NAD, buffers, and the compound to be evaluated; the contents of the reaction vessels are allowed to react for a sufficient amount of time under the appropriate incubation conditions; and the amount of NADH produced in each reaction vessel is monitored by methods known in the art; the amount of NADH produced by wild-type holo-enzyme is compared with the amount produced by the modified IMPDH polypeptide, to determine the relative sensitivity of the modified IMPDH polypeptides to the compound.

For example, the inhibitory effect of MPA on the functional activity (e.g., NADH production) of the modified IMPDH multimers may be determined using a serial dilution method and a steady state enzyme kinetic method (S. F. Carr, et al. 1993 supra; B. Xiang, et al., 1996 supra).

2. Nucleic Acid Molecules that Encode Modified IMPDH Polypeptides

The present invention provides various isolated, and recombinant nucleic acid molecules having polynucleotide sequences that encode modified IMPDH polypeptides of the invention, herein referred to as “modified impdh polynucleotides sequences”. The present invention also provides polynucleotide fragments that encode the modified IMPDH polypeptides, and related polynucleotide molecules, such as polynucleotide sequences complementary to modified IMPDH or a part thereof, and those that hybridize to the nucleic acid molecules of the invention.

The modified impdh polynucleotides, also referred to herein as nucleic acid molecules of the invention, are preferably in isolated form, and include, but are not limited to, DNA, RNA, DNA/RNA hybrids, and related molecules, and fragments thereof. Specifically contemplated are genomic DNA, ribozymes, and antisense molecules, as well as nucleic acids based on an alternative backbone or including alternative bases, whether derived from natural sources or synthesized.

a.) Isolated Polynucleotide Sequences of the Invention

The present invention provides nucleic acid molecules having polynucleotide sequences that encode the modified IMPDH polypeptides, or fragments thereof, embodied in many forms, preferably in an isolated form. The impdh polynucleotide sequences of the invention may be isolated as naturally-synthesized polynucleotides or from any source whether natural, synthetic, semi-synthetic, or recombinant. Accordingly, the modified impdh polynucleotide sequences may be isolated from any species, particularly mammalian, including bovine, ovine, porcine, murine, equine, and preferably human. Standard methods can be employed for isolating impdh polynucleotides, see for example Molecular Cloning; A Laboratory Manual, 2^{nd} edition, Sambrook, Fritch, and Maniatis 1989, Cold Spring Harbor Press.

For example, the polynucleotide sequences that encode the modified IMPDH polypeptides may be generated by isolating a cDNA clone encoding wild-type IMPDH protein, and then using recombinant DNA technology to manipulate the cDNA clone to replace the subdomain sequence with a nucleotide sequence that encodes the substitution oligo-peptide. The recombinant DNA method may, for example, include PCR technology (U.S. Pat. No. 4,603,102).

b.) The Polynucleotide Sequences that Encode Modified IMPDH Polypeptides of the Invention

The present invention provides isolated nucleic acid molecules having a polynucleotide sequence that encodes the modified IMPDH polypeptides of the invention. For example, the present invention provides isolated nucleic acid molecules having sequence identity with any of the polynucleotide sequences that encode the modified IMPDH polypeptides having a substitute tri-peptide as described in SEQ ID NOS.: 40-44. Alternatively, the present invention provides isolated nucleic acid molecules having sequence identity with any of the polynucleotide sequences that encode the modified IMPDH polypeptides having a substitute tetra-peptide as described in SEQ ID NOS.:45-47.

c.) Nucleotide Sequences that Encode Fragments of the Modified IMPDH Polypeptides

The invention further provides nucleic acid molecules having polynucleotide sequences that encode portions or fragments of the modified IMPDH polypeptides. The size of the polynucleotide fragment will be determined by its intended use. For example, the length of the fragment to be used as a nucleic acid probe or PCR primer is chosen to obtain a relatively small number of false positives during probing or priming. Alternatively, a fragment of the modified impdh polynucleotide sequences may be used to construct a recombinant fusion gene having a modified impdh polynucleotide sequence fused to a different sequence, such as a nucleotide sequence that encodes a histidine-tag to facilitate isolation and/or purification of the expressed polypeptide.

The probes, primers, and fragments of the present invention are useful for a variety of molecular biology techniques including, for example, hybridization screens of genomic or cDNA libraries, or detection and quantification of mRNA species as a means for analysis of gene expression. Preferably, the probes and primers are DNA. A probe or primer length of at least 15 base pairs is suggested by theoretical and practical considerations (Wallace and Miyada 1987 in: Methods in Enzymology 152:432-442, Academic Press). The probes and primers of this invention can be prepared by methods well known to those skilled in the art (see e.g., Sambrook et al., 1989 supra). In a preferred embodiment, the probes and primers are synthesized by polymerase chain reaction as disclosed in U.S. Pat. No. 4,683,202.

One embodiment of the present invention provides nucleic acid primers that are complementary to the modified impdh polynucleotide sequences, which allow the specific amplification of nucleic acid molecules of the invention or of any specific parts thereof.

d.) Sequences Complementary to the Modified impdh Sequences

The present invention includes polynucleotide sequences that are complementary to the nucleotide sequences of the invention, e.g., those described in SEQ ID NOS.: 40-47.

e.) Sequences Capable of Hybridizing to impdh Sequences

The present invention provides nucleic acid molecules having polynucleotide sequences that will selectively hybridize to modified impdh polynucleotide sequences under high stringency hybridization conditions, such as sequences described in SEQ ID NO.:40-47. Typically, hybridization under standard high stringency conditions will occur between two nucleic acid molecules that are complementary with each other (e.g., 100% exact complementarity), or two nucleic acid molecules that are nearly complementary with each other (e.g., about 70% to 99% identical, such as homologous sequences). It is readily apparent to one skilled in the art that the high stringency hybridization between nucleic acid molecules depends upon, for example, the degree of homology, the stringency of hybridization, and the length of hybridizing strands.

The high stringency hybridization conditions that disfavor non-homologous base pairing are well known in the art. Typically, high stringency hybridization conditions comprise hybridizing at 50° C. to 65° C. in 5× SSPE and 50% formamide, and washing at 50° C. to 65° C. in 0.5× SSPE. Typical low stringency conditions comprise hybridizing at 35° C. to 37° C. in 5× SSPE and 40% to 45% formamide and washing at 42° C. in 1-2× SSPE.

The conditions and formulas for high stringency hybridization methods are well known in the art and can be readily obtained from Molecular Cloning; A Laboratory Manual, 2^{nd} edition, Sambrook, Fritch, and Maniatis 1989, Cold Spring Harbor Press.

f.) Codon-usage Variants that Encode the Modified IMPDH Polypeptides

It may be advantageous to generate codon-usage variants that are altered from the disclosed nucleotide sequences, yet do not alter the encoded amino acid sequence of the modified IMPDH polypeptides. For example, the codons may be selected to optimize the level of production of the modified impdh transcript or the modified IMPDH polypeptide in a particular prokaryotic or eukaryotic expression host, in accordance with the frequency of codon utilized by the host cell. Alternative reasons for altering the nucleotide sequence encoding a modified IMPDH polypeptide include the production of RNA transcripts having more desirable properties, such as an increased half-life. A multitude of variant nucleotide sequences that encode the modified IMPDH polypeptides may be isolated, as a result of the degeneracy of the genetic code. Accordingly, the present invention contemplates selecting every possible triplet codon to produce every possible combination of nucleotide sequences that encode the disclosed amino acid sequence of the modified IMPDH polypeptides. One embodiment of the present invention provides isolated nucleotide sequences that vary from the sequences as described in SEQ ID NO.: 40-47 such that each variant nucleotide sequence encodes a polypeptide having sequence identity with the amino acid sequences of the modified IMPDH polypeptides, as described in SEQ ID NOs.: 20, 22, 27, 29, 30, 34, 36, or 38 respectively.

The amino acid coding sequence is as follows:

| | | One Letter | |
|---|---|---|---|
| Amino Acid | Symbol | Symbol | Codons |
| Alanine | Ala | A | GCU, GCC, GCA, GCG |
| Cysteine | Cys | C | UGU, UGC |
| Aspartic Acid | Asp | D | GAU, GAC |
| Glutamic Acid | Glu | E | GAA, GAG |
| Phenylalanine | Phe | F | UUU, UUC |
| Glycine | Gly | G | GGU, GGC, GGA, GGG |
| Histidine | His | H | CAU, CAC |
| Isoleucine | Ile | I | AUU, AUC, AUA |
| Lysine | Lys | K | AAA, AAG |
| Leucine | Leu | L | UUA, UUG, CUU, CUC, CUA, |
| | | | CUG |
| Methionine | Met | M | AUG |
| Asparagine | Asn | N | AAU, AAC |
| Proline | Pro | P | CCU, CCC, CCA, CCG |
| Glutamine | Gln | Q | CAA, CAG |
| Arginine | Arg | R | CGU, CGC, CGA, CGG, AGA, |
| | | | AGG |
| Serine | Ser | S | UCU, UCC, UCA, UCG, AGU, |
| | | | AGC |
| Threonine | Thr | T | ACU, ACC, ACA, ACG |
| Valine | Val | V | GUU, GUC, GUA, GUG |
| Tryptophan | Trp | W | UGG |
| Tyrosine | Tyr | Y | UAU, UAC |

g.) RNA Molecules that Encode the Modified IMPDH Polypeptides

The present invention provides isolated RNA molecules that encode the modified IMPDH polypeptides, or fragments thereof, as described in SEQ ID NOS.: 20-39. In particular, the RNA molecules of the invention may be isolated, full-length or partial mRNA molecules, or RNA oligomers that encode modified IMPDH polypeptides. The RNA molecules may be isolated as naturally-occurring molecules, or generated by recombinant DNA technology or chemical synthesis. The RNA molecules of the invention each include the nucleotide sequences encoding all or portions of the modified IMPDH polypeptides. The RNA molecules of the invention are useful as hybridizing nucleic acid probes for the detection of nucleotide sequences that encode the modified IMPDH polypeptides.

h.) Labeled Nucleic Acid Molecules Encoding the Modified IMPDH Polypeptides

The nucleic acid molecules having modified IMPDH polynucleotide sequences can be labeled with a detectable marker. The labeled IMPDH polynucleotide sequences may be used as hybridizable nucleic acid probes for the detection of nucleic acid molecules that encode modified IMPDH polypeptides. Examples of a detectable marker include, but are not limited to, a radioisotope, a fluorescent compound, a bioluminescent compound, a chemiluminescent compound, a metal chelator or an enzyme. Technologies for generating labeled DNA and RNA probes are well known in the art (Sambrook, et al., 1989 supra).

i.) Variants of Nucleotide Sequences Encoding the Modified IMPDH Polypeptides

The present invention provides modified impdh polynucleotide sequences that vary from the sequences described in SEQ ID NOS.: 40-47. The variant polynucleotide sequences may be isolated from naturally-occurring sources or generated by recombinant DNA technology. The variant polynucleotide sequences encode the modified IMPDH polypeptides described in SEQ ID NOS.: 20, 22, 27, 29, 30, 34, 36, or 38 respectively.

The modified impdh polynucleotide sequences encode modified IMPDH polypeptides that have conservative or non-conservative changes in the substitute tri- or tetra-peptide region. For example, a variant impdh polynucleotide sequence may encode a variant of the modified IMPDH polypeptide having conservative amino acid changes in the substitute oligo-peptide region, such as replacement of leucine with isoleucine. In another embodiment, an impdh polynucleotide variant may have nonconservative changes, such as replacement of glycine with a tryptophan.

A polynucleotide sequence can encode conservative amino acid substitutions without altering either the conformation or the function of the polypeptide. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered conservative in particular environments.

j.) Derivative Nucleic Acid Molecules that Encode the Modified IMPDH Polypeptides

The nucleic acid molecules of the invention also include peptide nucleic acids (PNAs), or derivative molecules such as phosphorothioate, phosphotriester, phosphoramidate, and methylphosphonate, that specifically bind to single-stranded DNA or RNA in a base pair-dependent manner (Zamecnik, P. C., et al., 1978 Proc. Natl. Acad. Sci. 75:280284; Goodchild, P. C., et al., 1986 Proc. Natl. Acad. Sci. 83:4143-4146).

PNA molecules comprise a nucleic acid oligomer to which an amino acid residue, such as lysine, and an amino group have been added. These small molecules, also designated anti-gene agents, stop transcript elongation by binding to their complementary (template) strand of nucleic acid (Nielsen, P. E., et al., 1993 Anticancer Drug Des 8:53-63). For example, reviews of methods for synthesis of DNA, RNA, and their analogues can be found in: Oligonucleotides and Analogues, eds. F. Eckstein, 1991, IRL Press, New York; Oligonucleotide Synthesis, ed. M. J. Gait, 1984, IRL Press, Oxford, England. Additionally, methods for antisense RNA technology are described in U.S. Pat. Nos. 5,194,428 and 5,110,802. A skilled artisan can readily obtain these classes of nucleic acid molecules using the polynucleotide sequences described herein, see for example Innovative and Perspectives in Solid Phase Synthesis (1992) Egholm, et al. pp 325-328 or U.S. Pat. No. 5,539,082.

3. Recombinant Nucleic Acid Molecules and Host Vector Systems Which Include Modified IMPDH Sequences

The present invention provides recombinant nucleic acid molecules, such as recombinant DNA molecules (rDNAs) that encode the modified IMPDH polypeptide sequences, or fragments thereof. As used herein, a rDNA molecule is a DNA molecule that has been subjected to molecular manipulation in vitro. Methods for generating rDNA molecules are well known in the art, for example, see Sambrook et al., Molecular Cloning (1989), and are useful for producing the modified IMPDH polypeptides.

The nucleic acid molecules of the invention may be recombinant molecules each comprising a modified impdh polynucleotide sequence linked to a different sequence. For example, the modified impdh polynucleotide sequence may be linked operatively to a vector to generate a recombinant vector.

a.) Vectors that Include the Modified impdh Sequences

The term vector includes, but is not limited to, plasmids, cosmids, and phagmids. An autonomously replicating vector typically refers to a nucleic acid molecule comprising a replicon that directs the replication of the rDNA within the appropriate host cell. The preferred vectors also include an expression control element, such as a promoter sequence, which enables transcription of the inserted modified impdh polynucleotide sequences and can be used for regulating the expression (e.g., transcription and/or translation) of an operably linked modified IMPDH sequence in an appropriate host cell such as E. coli. Prokaryote expression control elements are known in the art and include, but are not limited to, inducible promoters, constitutive promoters, secretion signals, enhancers, transcription terminators, and other transcriptional regulatory elements. Other expression control elements that are involved in translation are known in the art, and include the Shine-Delgamo sequence, and initiation and termination codons.

The preferred vector also includes at least one selectable marker gene that encodes a gene product that confers drug resistance, such as resistance to ampicillin, tetracyline, or kanamycin. Typically, a vector also comprises multiple endonuclease restriction sites that enable convenient insertion of exogenous DNA sequences.

The preferred vectors are expression vectors that are compatible with prokaryotic host cells. Prokaryotic cell expression vectors are well known in the art and are available from several commercial sources. Typical of such vectors is the pET24a expression vector which is used to express foreign genes in E. coli, includes the T7 RNA polymerase system, and confers resistance to kanamycin (Novagen, Inc., Madison, Wis.).

b.) Fusion Genes that Include the Modified impdh Sequences

A fusion gene is another example of recombinant molecules comprising a modified impdh polynucleotide sequence fused to a different sequence. For example, the modified impdh polynucleotide sequence may be fused to a tag sequence that encodes contiguous Histidine residues to facilitate isolation and/or purification of the expressed modified IMPDH polypeptide (Marshak, D. R., et al., 1996 in: Strategies for Protein Purification and Characterization pp 396).

Alternatively, a chimeric recombinant molecule includes the substitute oligo-peptide linked with impdh sequences that were each isolated from different sources. For example, the polynucleotide sequences that encode the N-terminal catalytic domain may be from a different source than the polynucleotide sequences that encodes the C-terminal domain. The N- and C-terminal domains of IMPDH may be from human (Collart and Hubermann 1988 supra), Chinese hamster (Natsutmeda and Carr 1993 supra) or other eukaryote organisms, or a prokaryote organism.

B. Host-vector Systems

Host cells harboring the nucleic acid molecules disclosed herein are also provided by the present invention. The invention provides a host-vector system comprising a suitable host cell introduced with the vectors, plasmids, phagmids, or cosmids comprising nucleotide sequences that encode the modified IMPDH polypeptides, or fragments thereof. The host cell can be either prokaryotic or eukaryotic. For example, many commercially-available strains of Escherichia coli are particularly useful for expression of foreign proteins. Examples of suitable eukaryotic host cells include a yeast cell, a plant cell, an insect cell, or an animal cell, such as a mammalian cell. A preferred embodiment provides a host-vector system comprising a recombinant Novagen pET24a (Novagen, Inc., Madison, Wis.) vector including the modified impdh polynucleotide sequences introduced into a E. coli BL21 (DE3) host cell (Novagen), which is useful, for example for the production of the modified IMPDH protein.

The recombinant DNA molecules of the present invention may be introduced into an appropriate cell host by well known methods that typically depend on the type of vector used and host system employed. For example, transformation of prokaryotic host cells by electroporation and salt treatment methods are typically employed, see, for example, Cohen et al., Proc Acad Sci USA (1972) 69:2110; and Maniatis et al., (1989) in: Molecular Cloning, A Laboratory Manual, Cold Spring Harbor Laboratory, Cold Spring Harbor, N.Y. Transformation of vertebrate cells with vectors containing rDNAs, electroporation, cationic lipid or salt treatment methods are typically employed (Graham et al., 1973 Virology 52:456; Wigler et al., 1979 Proc. Natl. Acad. Sci. USA 76:1373-76).

The cells introduced with the recombinant DNA molecules may be identified by well known techniques. For example, cells resulting from the introduction of the rDNA of the present invention can be cloned to produce single colonies. Cells from those colonies can be harvested, lysed and their DNA content examined for the presence of the rDNA using a method such as that described by Southern, J. Mol. Biol. (1975) 98:503, or Berent et al., Biotech. (1985) 3:208 or the proteins produced from the cell assayed via an immunological method.

The vector selected may be an expression vector for expression and production of the modified IMPDH polypeptides in the bacterial host cells. For example, vectors which direct high level expression of fusion proteins that are readily purified may be desirable. Such vectors include, but are not limited to, the multifunctional E. coli cloning and expression vectors such as BLUESCRIPT (Stratagene), in which the modified IMPDH coding sequence may be ligated into the vector in frame with sequences for the amino-terminal Met and the subsequent 7 residues of β-galactosidase so that a hybrid protein is produced; pIN vectors (Van Heeke & Schuster 1989 J. Biol Chem 264:5503-5509); and the like. The pGEX vectors (Promega, Madison Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the GST moiety at will.

In yeast host cells, such as Saccharomyces cerevisiae, a number of vectors containing constitutive or inducible promoters such as β-factor, alcohol oxidase and PGH may be used. For reviews, see F. Ausubel et al., 1989 in: Current Protocols in Molecular Biology, John Wiley & Sons, New York N.Y. and Grant et al., 1987 Methods in Enzymology 153:516-544.

In cases where plant expression vectors are used, the expression of a sequence encoding modified IMPDH polypeptides may be driven by any of a number of promoters. For example, viral promoters such as the 35S and 19S promoters of CaMV (Brisson et al., 1984 Nature 310:511-514) may be used alone or in combination with the omega leader sequence from TMV (Takamatsu et al., 1987 EMBO J. 6:307-311). Alternatively, plant promoters such as the small subunit of RUBISCO (Coruzzi et al., 1984 EMBO J 3:1671-1680; Broglie et al., 1984 Science 224:838-843); or heat shock promoters (Winter, J. and Sinibaldi, R. M. 1991 Results Probl. Cell. Differ. 17:85-105) may be used. These constructs can be introduced into plant cells by direct DNA transformation or pathogen-mediated transfection. For reviews of such techniques, see Hobbs, S. or Murry L E in: McGraw Yearbook of Science and Technology (1992) McGraw Hill New York N.Y., pp 191-196 or Weissbach and Weissbach (1988) Methods for Plant Molecular Biology, Academic Press, New York N.Y., pp 421-463.

An alternative expression system which could be used to express modified IMPDH polypeptides is an insect system. In one such system, Autographa califormica nuclear polyhedrosis virus (AcNPV) is used as a vector to express foreign genes in Spodoptera frugiperda cells or in Trichoplusia larvae. The modified IMPDH encoding sequence may be cloned into a nonessential region of the virus, such as the polyhedrin gene, and placed under control of the polyhedrin promoter. Successful insertion of the impdh nucleotide sequence will render the polyhedrin gene inactive and produce recombinant virus lacking coat protein. The recombinant viruses are then used to infect S. frugiperda cells or Trichoplusia larvae in which the modified IMPDH polypeptide is expressed (Smith et al., 1983 J. Virol 46:584; Engelhard, E. K. et al., 1994 Proc Nat Acad Sci 91:3224-7).

In mammalian host cells, a number of viral-based expression systems may be utilized. In cases where an adenovirus is used as an expression vector, a modified impdh coding sequence may be operably linked to an adenovirus vector including adenoviral late promoter (e.g., for transcription) and tripartite leader sequence (e.g., for translation). Insertion in a nonessential E1 or E3 region of the viral genome will result in available virus capable of expressing modified IMPDH in infected host cells (Logan and Shenk 1984 Proc Natl Acad Sci 81:3655-59). In addition, transcription enhancers, such as the rous sarcoma virus (RSV) enhancer, may be used to increase expression in mammalian host cells.

Specific initiation signals may also be required for efficient translation of a modified impdh sequence. These signals include the ATG initiation codon and adjacent sequences.

In cases where the modified impdh initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al., 1994 Results Probl Cell Differ 20:125-62; Bittner et al., 1987 Methods in Enzymol 153:516-544).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, W138, etc have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express modified IMPDH may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler, M. et al., 1977 Cell 11:223-32) and adenine phosphoribosyltransferase (Lowy, I. et al., 1980 Cell 22:817-23) genes which can be employed in tk-minus or aprt-minus cells, respectively. Also, antimetabolite, antibiotic or herbicide resistance can be used as the basis for selection; for example, dhfr which confers resistance to methotrexate (Wigler, M. et al., 1980 Proc Natl Acad Sci 77:3567-70); npt, which confers resistance to the aminoglycosides neomycin and G-418 (Colbere-Garapin, F. et al., 1981 J. Mol Biol 150:1-14) and als or pat, which confer resistance to chlorsulfuron and phosphinotricin acetyltransferase, respectively. Additional selectable genes have been described, for example, trpB, which allows cells to utilize indole in place of tryptophan, or hisD, which allows cells to utilize histinol in place of histidine (Hartman, S. C. and R. C. Mulligan 1988 Proc Natl Acad Sci 85:8047-51). Recently, the use of visible markers has gained popularity with such markers as anthocyanins, β-glucuronidase and its substrate, GUS, and luciferase and its substrate, luciferin, being widely used not only to identify transformants, but also to quantify the amount of transient or stable protein expression attributable to a specific vector system (Rhodes, C. A., et al., 1995 Methods Mol Biol 55:121-131).

C. Methods for Generating Modified IMPDH Polypeptides

The modified IMPDH polypeptides of the invention, and fragments thereof, can be generated by recombinant methods or chemical synthesis methods.

Recombinant methods are preferred if a high yield is desired. In general terms, the production of recombinant modified IMPDH polypeptides will involve using a host/vector system which typically involves the following steps. First, a nucleic acid molecule is obtained that encodes a modified IMPDH polypeptide, or a fragment thereof, such as any one of the polynucleotide sequences disclosed in SEQ ID NOs.: 40-47, or sequence variants as described above. The modified IMPDH-encoding nucleic acid molecule is then preferably inserted into an expression vector in operable linkage with suitable expression control sequences, as described above, to generate a recombinant expression vector including the modified IMPDH-encoding sequence. The expression vector is then introduced into a suitable host, by standard transformation methods, and the resulting transformed host is cultured under conditions that allow the in vivo production of the modified IMPDH polypeptide. For example, if expression of the modified impdh gene is under the control of an inducible promoter, then suitable growth conditions would include the appropriate inducer. The recombinant vector can integrate the modified impdh sequence into the host genome. Alternatively, the recombinant vector can maintain the modified impdh sequence extra-chromosomally, as part of an autonomously replicating vector. The modified IMPDH polypeptide, so produced, is isolated from the growth medium or directly from the cells; recovery and purification of the protein may not be necessary in some instances where some impurities may be tolerated. A skilled artisan can readily adapt an appropriate host/expression system known in the art for use with modified IMPDH-encoding sequences to produce modified IMPDH polypeptides (Cohen et al., 1972 Proc. Acad. Sci. USA 69:2110; and Maniatis et al., 1989 Molecular Cloning, A Laboratory Manual, Cold Spring Harbor Laboratory, Cold Spring Harbor, N.Y.). Examples of various protein purification methods can be found in Strategies for Protein Purification and Characterization (1996) pp 396, Marshak, D. R., et al. One embodiment provides modified IMPDH polypeptides purified using a series of up to three chromatographic methods, including anion exchange chromatography (Deutscher, M. P., 1990 in: Guide to Protein Purification: Methods in Enzymology, Academic Press, Inc., San Diego, Calif.), dye affinity chromatography (Deutscher, M. P., 1990 in: Guide to Protein Purification: Methods in Enzymology, Academic Press, Inc., San Diego, Calif.), IMP affinity chromatography (Ikegami, T., et al., 1987, Life Sciences 40: 2277-2282), and gel permeation chromatography (Deutscher, M. P., 1990 in Guide to Protein Purification: Methods in Enzymology, Academic Press, Inc., San Diego, Calif.).

The modified IMPDH polypeptides of the present invention can also be made by chemical synthesis. The principles of solid phase chemical synthesis of polypeptides are well known in the art and may be found in general texts relating to this area (Dugas, H. and Penney, C. 1981 in: Bioorganic Chemistry, Springer-Verlag, New York, pp 54-92). Modified IMPDH polypeptides may be synthesized by solid-phase methodology utilizing an Applied Biosystems 430A peptide synthesizer (Applied Biosystems, Foster City, Calif.) and synthesis cycles supplied by Applied Biosystems. Protected amino acids, such as t-butoxycarbonyl-protected amino acids, and other reagents are commercially available from many chemical supply houses.

D. Uses of the Modified IMPDH Polypeptides

The modified IMPDH polypeptides of the invention may be useful for a variety of purposes, including, but not limited to, their use as the ability to elicit the generation of antibodies, diagnostic and/or prognostic markers of diseases, and as targets for various therapeutic modalities, as further described below. The modified IMPDH polypeptides may also be used to identify and isolate ligands and other agents that bind to modified IMPDH. The modified IMPDH polypeptides, and fragments thereof, can be generated using standard peptide synthesis technology or recombinant DNA technology.

1. Antibodies that Recognize and Bind the Modified IMPDH Polypeptides

The peptides of the invention exhibit properties of modified IMPDH polypeptides, such as the ability to elicit the generation of antibodies that specifically bind an epitope associated with various domains of the modified and/or wild type IMPDH polypeptides. These antibodies can be used to identify and/or target cells that express modified IMPDH polypeptides. For example, these antibodies can be used to deliver conjugated toxins to cells that express wild-type or other forms of IMPDH, in order to modulate IMPDH activity or to directly kill cells expressing wild-type or other forms of IMPDH. The conjugated toxins include, but are not limited to, diphtheria toxin, cholera toxin, ricin or pseudomonas exotoxin. Alternatively, these antibodies can be used to identify agents that interact with wild-type IMPDH or modified IMPDH polypeptides/multimers. These antibodies can also be used in screening assays to identify competitive binding agents.

The invention provides antibodies (e.g., polyclonal, monoclonal, chimeric, immunologically active fragments, and humanized antibodies) that bind to the modified and/or wild type IMPDH polypeptides. The most preferred antibodies will selectively bind to a particular modified IMPDH polypeptide, and will not bind (or will weakly bind) to a different modified IMPDH polypeptide, or polypeptides that are not modified IMPDH polypeptides. Antibodies that are particularly contemplated include monoclonal and polyclonal antibodies as well as fragments thereof (e.g., recombinant proteins) containing the antigen binding domain and/or one or more complement determining regions of these antibodies. These antibodies can be from any source, for example, rabbit, sheep, rat, dog, cat, pig, horse, mouse, and human.

The invention also encompasses antibody fragments that specifically recognize a modified IMPDH polypeptide, or a fragment thereof. As used herein, an antibody fragment is defined as at least a portion of the variable region of the immunoglobulin molecule that binds to its target, i.e., the antigen binding region. Some of the constant region of the immunoglobulin may be included.

As will be understood by those skilled in the art, the regions or epitopes of a modified IMPDH polypeptide to which an antibody is directed may vary with the intended application. Antibodies that recognize other epitopes may be useful for the identification of modified IMPDH polypeptides on or within damaged or dying cells, for the detection of membrane-bound or secreted forms of modified IMPDH polypeptides or fragments thereof.

Various methods for the preparation of antibodies are well known in the art. For example, antibodies may be prepared by immunizing a suitable mammalian host using a modified IMPDH polypeptide, or fragment, in isolated or immunoconjugated form (Harlow, E. and Lane, D. 1989 in: Antibodies: a Laboratory Manual). In addition, fusion proteins comprising modified IMPDH polypeptides may also be used, such as a modified IMPDH polypeptide/GST-fusion protein. Cells expressing or overexpressing a modified IMPDH polypeptide may also be used for immunizations. Similarly, any cell engineered to express modified IMPDH polypeptides may be used. This strategy may result in the production of monoclonal antibodies with enhanced capacities for recognizing endogenous modified IMPDH polypeptide.

The present invention contemplates chimeric antibodies that comprise at least two antibody portions from different species, for example, a human and non-human portion. Chimeric antibodies are useful, as they are less likely to be antigenic to a human subject than antibodies with non-human constant regions and variable regions. The antigen combining region (variable region) of a chimeric antibody can be derived from a human source and the constant region of the chimeric antibody which confers biological effector function to the immunoglobulin can be derived from a non-human source. The chimeric antibody should have the antigen binding specificity of the prokaryotic antibody molecule and the effector function conferred by the eukaryotic antibody molecule.

In general, the procedures used to produce chimeric antibodies can involve the following steps:

a) identifying and cloning the correct immunoglobin gene segment encoding the antigen binding portion of the antibody molecule; this gene segment (known as the VDJ, variable, diversity and joining regions for heavy chains or VJ, variable, joining regions for light chains or simply as the V or variable region) may be in either the cDNA or genomic form;

b) cloning the gene segments encoding the constant region or desired part thereof;

c) ligating the variable region with the constant region so that the complete chimeric antibody is encoded in a form that can be transcribed and translated;

d) ligating this construct into a vector containing a selectable marker and gene control regions such as promoters, enhancers and poly(A) addition signals;

e) amplifying this construct in bacteria;

f) introducing this DNA into eukaryotic cells (transfection) most often mammalian lymphocytes;

g) selecting for cells expressing the selectable marker;

h) screening for cells expressing the desired chimeric antibody; and

k) testing the antibody for appropriate binding specificity and effector functions.

Chimeric antibodies of several distinct antigen binding specificities have been produced by protocols well known in the art, including anti-TNP antibodies (Boulianne et al., 1984 Nature 312:643); and anti-tumor antigens antibodies (Sahagan et al., 1986 J. Immunol. 137:1066). Likewise, several different effector functions have been achieved by linking new sequences to those encoding the antigen binding region. Examples of these include enzymes (Neuberger et al., 1984 Nature 312:604); immunoglobulin constant regions from another species and constant regions of another immunoglobulin chain (Sharon et al., 1984 Nature 309:364; Tan et al., 1985 J. ImmunoL. 135:3565-3567). Additionally, procedures for modifying antibody molecules and for producing chimeric antibody molecules using homologous recombination to target gene modification have been described (Fell et al., 1989 Proc. Natl. Acad. Sci. USA 86:8507-8511).

The amino acid sequence of modified IMPDH polypeptides may be used to select specific regions of the modified IMPDH polypeptide for generating antibodies. For example, hydrophobicity and hydrophilicity analyses of a modified IMPDH polypeptide may be used to identify hydrophilic regions in the modified IMPDH polypeptide. Regions of the modified IMPDH polypeptide that show immunogenic structure, as well as other regions and domains, can readily be identified using various other methods known in the art (Rost, B., and Sander, C. 1994 Protein 19:55-72), such as Chou-Fasman, Gamier-Robson, Kyte-Doolittle, Eisenberg, Karplus-Schultz or Jameson-Wolf analysis. Fragments containing these residues are particularly suited for generating specific classes of antibodies.

Methods for preparing a protein for use as an immunogen and for preparing immunogenic conjugates of a protein with a carrier such as BSA, KLH, or other carrier proteins are well known in the art. In some circumstances, direct conjugation using, for example, carbodiimide reagents may be used; in other instances linking reagents such as those supplied by Pierce Chemical Co., Rockford, Ill., may be effective. Administration of a modified IMPDH immunogen is conducted generally by injection over a suitable time period and with use of a suitable adjuvant, as is generally understood in the art. During the immunization schedule, titers of antibodies can be taken to determine adequacy of antibody formation.

While the polyclonal antisera produced in this way may be satisfactory for some applications, for pharmaceutical compositions, monoclonal antibody preparations are preferred. Immortalized cell lines which secrete a desired monoclonal antibody may be prepared using the standard method of Kohler and Milstein (Nature 256: 495-497) or other techniques as described in Monoclonal Antibodies; A Manual of Techniques, CRC press, Inc., Boca Raton, Fla. (1987) ed., Zola. The immortalized cell lines secreting the desired antibodies are screened by immunoassay in which the antigen is the modified IMPDH polypeptide or fragment thereof. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal antibodies of the invention or the polyclonal antisera (e.g., Fab, F(ab′)₂, Fv fragments, fusion proteins) which contain the immunologically significant portion (i.e., a portion that recognizes and binds a modified IMPDH polypeptide) can be used as antagonists, as well as the intact antibodies. Humanized antibodies directed against a modified IMPDH polypeptide are also useful. As used herein, a humanized antibody is an immunoglobulin molecule which is capable of binding to a modified IMPDH polypeptide and which comprises a FR region having substantially the amino acid sequence of a human immunoglobulin and a CDR having substantially the amino acid sequence of non-human immunoglobulin or a sequence engineered to bind a modified IMPDH polypeptide. Methods for humanizing murine and other non-human antibodies by substituting one or more of the non-human antibody CDRs for corresponding human antibody sequences are well known (see for example, Jones et al., 1986 Nature 321: 522-525; Riechmnan et al., 1988 Nature 332: 323-327; Verhoeyen et al., 1988 Science 239: 1534-1536; Carter et al., 1993 Proc. Natl. Acad. Sci. USA 89: 4285; and Sims et al., 1993 J. Immunol. 151: 2296). Use of immunologically reactive fragments, such as the Fab, Fab′, or F(ab′)₂ fragments is often preferable, especially in a therapeutic context, as these fragments are generally less immunogenic than the whole immunoglobulin. Further, bi-specific antibodies specific for two or more epitopes may be generated using methods generally known in the art. Further, antibody effector functions may be modified so as to enhance the therapeutic effect of the antibodies of the invention. For example, cysteine residues may be engineered into the Fc region, permitting the formation of interchain disulfide bonds and the generation of homodimers which may have enhanced capacities for internalization, ADCC and/or complement-mediated cell killing (see, for example, Caron et al., 1992 J. Exp. Med. 176: 1191-1195; Shopes, 1992 J. Immunol. 148: 2918-2922; Liu et al., 1998 Cancer Research 58:4055-4060). Homodimeric antibodies may also be generated by cross-linking techniques known in the art (e.g., Wolff et al., Cancer Res. 53: 2560-2565). The invention also provides pharmaceutical compositions having the monoclonal antibodies or anti-idiotypic monoclonal antibodies of the invention.

The antibodies or fragments may also be produced, using current technology, by recombinant means. Regions that bind specifically to the desired regions of the modified IMPDH polypeptide can also be produced in the context of chimeric or CDR grafted antibodies of multiple species origin. The invention includes an antibody, e.g., a monoclonal antibody which competitively inhibits the immunospecific binding of any of the monoclonal antibodies of the invention to a modified IMPDH polypeptide.

Alternatively, methods for producing fully human monoclonal antibodies, include phage display and transgenic methods, are known and may be used for the generation of human mAbs (for review, see Vaughan et al., 1998 Nature Biotechnology 16: 535-539). For example, fully human monoclonal antibodies may be generated using cloning technologies employing large human Ig gene combinatorial libraries (i.e., phage display) (Griffiths and Hoogenboom, “Building an in vitro immune system: human antibodies from phage display libraries” in: Protein Engineering of Antibody Molecules for Prophylactic and Therapeutic Applications in Man, Clark, M. (Ed.), Nottingham Academic, pp 45-64 (1993); Burton and Barbas, “Human Antibodies from combinatorial libraries” Id., pp 65-82). Fully human monoclonal antibodies may also be produced using transgenic mice engineered to contain human immunoglobulin gene loci as described in PCT Patent Application WO98/24893, Jakobovits et al., published Dec. 3, 1997 (see also, Jakobovits, 1998, Exp. Opin. Invest. Drugs 7:607-614). This method avoids the in vitro manipulation required with phage display technology and efficiently produces high affinity authentic human antibodies.

The antibody or fragment thereof of the invention may be labeled with a detectable marker or conjugated to a second molecule, such as a therapeutic agent (e.g., a cytotoxic agent) thereby resulting in an immunoconjugate. For example, the therapeutic agent includes, but is not limited to, an anti-tumor drug, a toxin, a radioactive agent, a cytokine, a second antibody or an enzyme. Further, the invention provides an embodiment wherein the antibody of the invention is linked to an enzyme that converts a prodrug into a cytotoxic drug.

Examples of cytotoxic agents include, but are not limited to ricin, ricin A-chain, doxorubicin, daunorubicin, taxol, ethiduim bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, arbrin A chain, modeccin A chain, alpha-sarcin, gelonin, mitogellin, retstrictocin, phenomycin, enomycin, curicin, crotin, calicheamicin, sapaonaria officinalis inhibitor, maytansinoids, glucocorticoid and other chemotherapeutic agents, as well as radioisotopes such as ²¹²Bi, ¹³¹I, ¹³¹In, ⁹⁰Y, and ¹⁸⁶Re. Suitable detectable markers include, but are not limited to, a radioisotope, a fluorescent compound, a bioluminescent compound, chemiluminescent compound, a metal chelator or an enzyme. Antibodies may also be conjugated to a cell killing or inhibiting, pro-drug activating enzyme capable of converting the pro-drug to its active form. See, for example, U.S. Pat. Nos. 4,952,394 and 5,632,999.

Additionally, a recombinant protein of the invention comprising the antigen-binding region of any of the monoclonal antibodies of the invention can be made. In such a situation, the antigen-binding region of the recombinant protein is joined to at least a functionally active portion of a second protein having therapeutic activity. The second protein can include, but is not limited to, an enzyme, lymphokine, oncostatin or toxin. Suitable toxins include those described above.

Techniques for conjugating or joining therapeutic agents to antibodies are well known (see, eg, Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

2. Uses of the Antibodies that Recognize and Bind Modified IMPDH Polypeptides

The modified IMPDH antibodies of the invention may be particularly useful in diagnostic assays, imaging methodologies, and therapeutic methods in the management of cancer or other proliferative-type diseases. Such assays generally comprise one or more antibodies capable of recognizing and binding a modified IMPDH polypeptide, and include various immunological assay formats well known in the art, including but not limited to various types of precipitation, agglutination, complement fixation, radioimmunoassays (RIA), enzyme-linked immunosorbent assays (ELISA), enzyme-linked immunofluorescent assays (ELIFA) (Liu, H., et al. 1998 Cancer Research 58: 4055-4060), immunohistochemical analysis and the like.

Antibodies of the invention may also be used in methods for purifying modified IMPDH polypeptides and for isolating related molecules such as wild-type and mutant IMPDH polypeptides.

For example, in one embodiment, the method of purifying protein comprises incubating a modified IMPDH antibody, which has been coupled to a solid matrix, with a lysate or other solution containing IMPDH under conditions which permit the IMPDH antibody to bind to IMPDH polypeptides; washing the solid matrix to eliminate impurities; and eluting the IMPDH polypeptides from the coupled antibody. Additionally, IMPDH antibodies may be used to isolate IMPDH-positive cells using cell sorting and purification techniques. The presence and amount of IMPDH polypeptides on diseased cells (alone or in combination with other cell surface markers) may be used to distinguish and isolate diseased cells from other cells.

3. Cells that Express the Modified IMPDH Polypeptides

The ability to generate large quantities of relatively pure modified IMPDH-positive cells which can be grown in tissue culture or as a xenograft in animal models (e.g., SCID or other immune deficient mice) provides many advantages, including, for example, permitting the evaluation of various transgenes or candidate therapeutic compounds on the growth or other phenotypic characteristics of a relatively homogeneous population of diseased cells. Additionally, this feature of the invention also permits the isolation of highly enriched preparations of nucleic acid molecules that encode the modified IMPDH polypeptides; the nucleic acid molecules can be enriched in quantities sufficient for various molecular manipulations. For example, large quantities of such nucleic acid preparations will assist in the identification of rare genes with biological relevance to IMPDH-associated disease progression.

Another valuable application of this aspect of the invention is the ability to isolate, analyze and experiment with relatively pure preparations of viable modified IMPDH-positive tumor cells cloned from individual patients with primary, locally advanced or metastatic disease. In this way, for example, an individual patient's diseased cells that are modified IMPDH-positive may be expanded from a limited biopsy sample and then tested for the presence of diagnostic and prognostic genes, proteins, chromosomal aberrations, gene expression profiles, or other relevant genotypic and phenotypic characteristics, without the potentially confounding variable of contaminating cells. In addition, such cells may be evaluated for neoplastic aggressiveness and metastatic potential in animal models. Similarly, vaccines and cellular immunotherapeutics may be created from such cell preparations.

Use of immunologically reactive fragments, such as the Fab, Fab′, or F(ab′)₂ fragments is often preferable, especially in a therapeutic context, as these fragments are generally less immunogenic than the whole immunoglobulin. The invention also provides pharmaceutical compositions having the monoclonal antibodies or anti-idiotypic monoclonal antibodies of the invention.

4. Uses for Modified IMPDH Polypeptides in Drug Discovery Strategies

a.) Uses for the Modified IMPDH as Target Polypeptides

The modified IMPDH polypeptides of the invention are useful as starting points for structure-based drug design strategies. These strategies involve using information about the interaction of a target polypeptide or protein, such as a modified IMPDH polypeptide, and an agent that binds the target polypeptide. Typically, the goal of such drug design strategies is to identify an agent, or agents that bind to the target polypeptide and modulate the activity of the target polypeptide, such as activation or inhibition of the target polypeptide. Another goal is to alter the identified agent in order to develop a therapeutic agent that has improved properties, such as increased binding affinity for the target polypeptide, increased selectivity for the target polypeptide, or increased suitability for administration to a mammalian subject (e.g., a human patient). These agents may be useful for treating afflictions associated with abnormal cellular expression of IMPDH proteins, such as such as immune system diseases.

The structure-based drug design strategies can involve using information about the 3-dimensional structure of the target polypeptide complexed with the agent. For example, analysis of the X-ray crystal structure of the target polypeptides complexed with an inhibitor can provide information about the specific amino acid residues within the target polypeptide that interact and/or bind to the agent. Furthermore, it is advantageous to obtain crystal structures of a target polypeptide that exhibits the functional activity of the wild-type protein of interest. Oftentimes, the agent is quite small, therefore the technical challenge is to obtain a crystal structure of a functionally active target polypeptide that is complexed with the agent, at a level of resolution that is high enough to resolve the agent and its interaction with the target polypeptide.

The present invention provides the discovery that the modified IMPDH-DKT polypeptide is useful for obtaining protein crystal structures of a functionally active IMPDH multimer that is complexed with MPA, at a higher resolution level compared to crystal structures of wild-type IMPDH bound to MPA, obtained previously by other researchers. In particular, the present invention provides the discovery that the crystal structure of a homo-multimer comprising four subunits of modified human type II IMPDH-DKT and complexed with one molecule of MPA has been resolved at the 2.0 Angstrom level. In contrast, the crystal structure of homo-multimers of wild-type IMPDH from Chinese hamster complexed with MPA has been resolved at the 2.6 Angstrom level by M. D. Sintchak, et al (1996 Cell 85:921-930).

The higher level of resolution of the homo-multimer comprising modified IMPDH-DKT is due to the fact that each modified IMPDH-DKT polypeptide is shorter than the wild-type IMPDH polypeptides. In particular, the modified IMPDH-DKT polypeptides have a portion of the subdomain region, measuring 133 amino acid residues in length, replaced with a shorter substitute DKT tri-peptide (e.g., single-letter amino acid code). Thus, the modified IMPDH-DKT polypeptides are only 384 amino acid residues in length (FIG. 4), compared to the wild-type type II IMPDH which is 514 amino acid residues long (FIG. 2). Further, the modified IMPDH polypeptides exhibit the functional activity of wild-type IMPDH (e.g., catalyzes NADH production) and bind to MPA. Thus, the modified IMPDH-DKT polypeptide is an ideal candidate target polypeptide for use in a structure-based drug design strategy to discover agents that bind modified IMPDH-DKT and wild-type IMPDH.

b.) Methods for Detecting and Identifying Agents that Bind Modified IMPDH

The present invention further provides methods for detecting and identifying agents that interact with the modified IMPDH polypeptides and/or multimers of the invention. The agents that interact with the modified IMPDH polypeptides or multimers of the invention may or may not cause a change in the activity of the modified IMPDH multimer, such as inhibition or stimulation. Accordingly, one embodiment of the present invention provides methods for identifying agents that interact with the modified IMPDH polypeptides or multimers. A further embodiment provides methods for identifying agents that affect the activity of the modified IMPDH multimers, such as agonists and antagonists.

The general method for identifying candidate agents that interact with or bind to modified IMPDH polypeptides or multimers comprises the following steps. Contacting the modified IMPDH polypeptides or multimer with the candidate agent, and incubating the contacted modified IMPDH polypeptides or multimers under conditions that allow interaction of the modified IMPDH polypeptides or multimers with the candidate agent, and detecting the interaction of the modified IMPDH polypeptides or multimers with the candidate agent by any suitable means. These methods may be performed in vivo or in vitro. Additionally, these methods may be adapted to automated procedures such as a PANDEX.RTM (Baxter-Dade Diagnostics) system, allowing for efficient high-volume screening of candidate agents.

The modified IMPDH polypeptides that may be used in the methods of the invention include, but are not limited to, an isolated modified IMPDH polypeptide, a fragment of a modified IMPDH polypeptide, a cell that has been altered to express a modified IMPDH polypeptide, or a fraction of a cell that has been altered to express a modified IMPDH polypeptide. These modified IMPDH polypeptides and fragments thereof, may associate with each other to form multimers.

The in vitro methods for identifying and detecting interaction between modified IMPDH polypeptides or multimers and the candidate agent include gel retardation assays, immunodetection, and biochip technologies can be adopted for use with the modified IMPDH polypeptides or multimers. Other methods include fluorescence titration (Freifelder, D., 1982 in: Physical Biochemistry: Applications to Biochemistry and Molecular Biology, W. H. Freeman & Co., San Francisco, Calif.) and titration calorimetry (Wiseman, T., et. al., 1989, Anal. Biochem. 179: 131-137). The in vitro methods also include monitoring the ratio of folded protein to unfolded protein, by monitoring sensitivity of the protein to a protease, or amenability to binding of the protein by a specific antibody against the folded state of the protein, or binding to chaperon protein, or binding to any suitable surface (U.S. Pat. No. 5,585,277). A skilled artisan can readily employ numerous art-known techniques for determining whether a particular agent binds to a modified IMPDH polypeptide or multimer.

Alternatively, an in vivo method for identifying and detecting interaction between candidate agents and modified IMPDH polypeptides or multimers can be performed in a whole cell assay using a yeast two-hybrid system (Fields, S. and Song, O. 1989 Nature 340:245-246). The yeast two-hybrid system can be used to screen cDNA expression libraries (G. J. Hannon, et al. 1993 Genes and Dev. 7: 2378-2391), random aptmer libraries (J. P. Manfredi, et al. 1996 Molec. And Cell. Biol. 16: 4700-4709), or semi-random (M. Yang, et al. 1995 Nucleic Acids Res. 23: 1152-1156) aptmer libraries for ligands that interact/bind with the modified IMPDH polypeptide. The interaction/binding between the modified IMPDH polypeptide or multimers and the agent can be detectable by expression of a reporter gene, such as lacZ.

The agents can be, for example, a ligand which is typically a polypeptide, a nucleic acid molecule, an organic molecule, vitamin derivatives, or a metal. A skilled artisan can readily recognize that there is no limit as to the structural nature of the agents used in the present screening methods. The agents can be synthetic or naturally-occurring compounds, such as cellular constituents. The cellular extracts tested in the methods of the present invention can be, as examples, aqueous extracts of cells or tissues, organic extracts of cells or tissues or partially purified cellular fractions.

The polypeptide agents can be generated using standard solid phase or solution phase peptide synthesis methods, as is known in the art. In addition, the nucleic acid molecules encoding these peptides may be generated using standard recombinant DNA technology or synthesized using commercially-available oligonucleotide synthesis instrumentation.

The amino acid sequence of the polypeptide agents can be chosen based on the structure of the modified IMPDH polypeptides or multimers. Small polypeptides can also serve as competitive inhibitors of assembly of modified IMPDH polypeptide into a modified IMPDH multimer.

The antibody agents can be immunoreactive with selected domains or regions of the modified IMPDH polypeptides. In general, antibodies are obtained by immunization of suitable mammalian subjects with peptides, containing as antigenic regions, those portions of the modified IMPDH polypeptides intended to be targeted by the antibodies.

Agents that are assayed in the methods described above can be randomly selected, or rationally selected or designed. As used herein, an agent is said to be randomly selected when the agent is chosen randomly without considering the specific sequences of the modified IMPDH polypeptide. An example of randomly selected agents is the use of a chemical library or a peptide combinatorial library, or a growth broth of an organism or plant extract.

As used herein, an agent is said to be rationally selected or designed when the agent is chosen on a nonrandom basis that takes into account the sequence of the target polypeptide and/or the conformation of the target polypeptide. Agents can be rationally selected or rationally designed by utilizing the amino acid sequences that make up the modified IMPDH polypeptides.

The agents that interact with the modified IMPDH multimers can be tested for the ability to modulate the functional activity of modified IMPDH multimers using a cell-free assay system or a cellular assay system. For example, agents that interact with the modified IMPDH multimers may inhibit or increase the catalytic conversion of NAD to NADH, as detected by a relative change in the level of NADH produced compared to wild-type IMPDH multimer or holo-enzyme Carr, S. F., et al., 1993 supra; Xiang, B., et al., 1996 supra).

As used herein, an agent is said to antagonize the activity of the modified IMPDH multimers when the agent reduces the activity of the modified IMPDH multimers, such as reduces the level of NADH produced. The preferred antagonist will selectively antagonize modified IMPDH multimers, not affecting any other cellular proteins or multimers. Further, the preferred antagonist will reduce the activity of the modified IMPDH multimers by more than 50%, more preferably by more than 90%, most preferably eliminating all activity of the modified IMPDH multimers.

As used herein, an agent is said to agonize the activity of the modified IMPDH multimers when the agent increases the activity of the modified IMPDH multimers, such as increases the level of NADH produced. The preferred agonist will selectively agonize modified IMPDH multimers, not affecting any other cellular proteins or multimers. Further, the preferred agonist will increase the activity of the modified IMPDH multimers by more than 50%, more preferably by more than 90%, most preferably more than doubling the activity of the modified IMPDH multimers.

4. Diagnostic uses of Modified IMPDH Polypeptides

There are multiple diagnostic uses of the modified IMPDH polypeptides. For example, the modified IMPDH polypeptides provide methods for diagnosing in a subject, such as an animal or a human subject, a disease or disorder associated with the presence of an abnormal amount of IMPDH polypeptides or proteins. In one embodiment, the method comprises quantitatively determining the amount of IMPDH protein in a suitable biological test sample using any one or a combination of the antibodies of the invention. Then the amount of IMPDH protein, so determined in the test sample, can be compared with the amount in a biological sample from a subject having normal amounts of IMPDH protein. The presence of a measurably different amount of IMPDH in the test sample compared to the amount from a normal sample may indicate the presence of the disorder.

A suitable biological sample includes blood, serum, cells, tissue, or a swab from nose, ear or throat. The amount of modified IMPDH polypeptides can be determined by methods well known in the art, such as immunoassay methods.

Alternatively diagnostic methods include using the nucleic acid molecules of the invention. For example, the amount of such sequences present within a suitable biological test sample may be determined by means of a molecular biological assay to determine the amount of nucleic acid molecules having nucleotide sequences complementary to the modified IMPDH sequences of the invention. The presence of a measurably different amount of nucleic acid molecules having the IMPDH sequences in the test sample compared to the amount from a normal sample may indicate the presence of the disorder. A suitable biological sample includes blood, serum, cells, tissue, or a swab from nose, ear or throat. The amount of nucleic acid molecules having modified IMPDH polynucleotide sequences can be determined by methods well known in the art, such as hybridization methods.

Generally, such a diagnostic method includes the following steps. Obtaining nucleic acid molecules from the biological test sample, contacting these test nucleic acid molecules with the nucleic acid molecule of the present invention under conditions that allow hybridization of the complementary sequences of the test nucleic acid molecules and the nucleic acid molecules of the invention, and detecting the presence of hybridized nucleic acid molecules. The presence of nucleotide sequences in a test sample that are complementary to the polynucleotide sequences of the invention, or a measurably different level of such a sequence, in comparison to the levels in a normal or “control” sample, may be indicative of a sample having the gene sequence of the invention. Here, complementary nucleic acid sequences are those that have relatively little sequence divergence and that are capable of hybridizing to the sequences disclosed herein under standard conditions.

A variety of hybridization methods are known that can be used in to detect the amount of nucleic acid molecules having the IMPDH sequences, including diagnostic assays such as those described in Falkow et al., U.S. Pat. No. 4,358,535. Other suitable variations of hybridization methods are available for use in the detection of nucleic acids. These include, for example, in situ hybridization, Southern blotting and Northern blotting.

The in situ hybridization methods generally involve contacting a target nucleic acid molecule located within one or more cells or tissue with a detectable nucleic acid probe having the IMPDH sequences of the invention. The cells or tissue samples may be primary samples obtained from the subject or cells grown in tissue culture. As is well known in the art, the cells are prepared for hybridization by fixation (e.g., chemical fixation), and placed in conditions that permit hybridization of the detectable probe with nucleic acids located within the fixed cell or tissue.

Alternatively, the presence and/or amount of target nucleic acids molecules having the IMPDH sequences may be determined by Southern (e.g., DNA) or Northern (e.g., RNA) blot methods. These methods involve isolating nucleic acid molecules from a test cell or tissue sample, separating the isolated nucleic acid molecules based according to size, immobilizing the separated nucleic acid molecules onto a solid matrix, and contacting the immobilized nucleic acid molecules with a detectable nucleic acid probe having the IMPDH sequence of the invention. The nucleic acid molecules can be isolated using methods such as cesium chloride gradient centrifugation, chromatography (e.g., ion, affinity, magnetic), phenol extraction and the like. The isolated nucleic acid molecules can be separated according to size using methods, including electrophoretic separation.

The nucleic acid molecules of the invention may be detected in a biological test sample using PCR technology (U.S. Pat. No. 4,603,102; incorporated herein by reference). The PCR method involves isolating nucleic acid molecules from a biological test sample, contacting the test nucleic acid molecules with two nucleic acid primers having sequences complementary to the IMPDH sequences of the invention, and incubating the test nucleic acid molecules and primers under conditions which allow for hybridization and polymerization to occur. Typically, a pair of primers, one corresponding to the 5′ flanking region and the other corresponding to the 3′ flanking region, are used to detect the presence and the amount of nucleic acid molecules of the invention in a test sample.

The following examples are presented to illustrate the present invention and to assist one of ordinary skill in making and using the same. The examples are not intended in any way to otherwise limit the scope of the invention.

### EXAMPLES

### Example 1

The following provides a description of the methods used to produce and isolate the human, type I and type II, modified IMPDH polypeptides of the invention.

A) Generating the Nucleotide Sequences Encoding Wild-type, Human IMPDH

PCR amplification was used to generate full-length, human impdh type I and II cDNAs from RNA isolated from PHA-activated human peripheral blood leukocytes. The primers used to amplify the wild-type impdh cDNAs include the following:

5′ primer, wild-type, type I, human impdh; SEQ ID NO.:50:

5′-CTA CGT CAT ATG GCT GAC TAC CTG ATC AGC GGC-3′

3′ primer, wild-type, type I, human impdh; SEQ ID NO.:51:

5′-CGA TGT AAG CTT TCA GTA CAG CCG CTT TTC GTA AGA G-3′

5′ primer, wild-type, type II, human impdh; SEQ ID NO.: 52:

5′-CTA CGT CAT ATG GCC GAC TAC CTG ATT AGT GGG-3′

3′ primer, wild-type, type II, human impdh; SEQ ID NO.: 53:

5′-CGA TGT AAG CTT TCA GAA AAG CCG CTT CTC ATA CG-3′

The 5′ primers included nucleotide sequences that are complementary to the N-terminal sequences of wild type impdh, which was modified to include an Nde I restriction site. The 3′ primers included nucleotide sequences that are complementary to the C-terminal sequences of wild-type impdh.

The PCR products were subcloned into the vector pET24a(+) (Novagen, Madison, Wis.) and transformed into DH5alpha F′ (IQ) competent cells (Gibco). The sequences were verified by nucleotide sequencing and compared with the reported genomic sequences (Zimmermann, A. G., et al., 1995 J. Biol. Chem. 270:6808-6814; Glesne, D. A. and Huberman, E. 1994 Biochem. Biophys. Res. Comm. 205:537-544).

The full-length, human, type I impdh cDNAs, which were generated as described above, were used as templates to generate PCR-amplified nucleotide sequences encoding IMPDH-DKT (SEQ ID NO:44). The full-length, human, type II impdh cDNAs were used as templates to generate PCR-amplified nucleotide sequences encoding the various tri- and tetra-peptide IMPDH, including IMPDH-DKT, -SPS, -GSG, -SPT, -AGRP, and -NSPL (examples include SEQ ID NOS:40-43, and 45-47). The primers used to amplify the various modified impdh sequences included the following:

5′ primer, N-terminal end, type II, impdh; SEQ ID NO.:54

5′-ggaattccatATGGCCGACTACCTG-3′

3′ primer, type II, DKT-IMPDH; SEQ ID NO.:55

5′-GGTCTTGTCatatttcttcactttccgaac-3′

3′ primer, type II, SPS-IMPDH; SEQ ID NO.: 56

5′-GCTCGGAGAatatttcttcactttccgaac-3′

3′ primer, type II, GSG-IMPDH; SEQ ID NO.:57

5′-GcCGGAACCatatttcttcactttccgaac-3′

3′ primer, type II, SPTQ-IMPDH; SEQ ID NO.: 58

5′-CTGAGTCGGAGAatatttcttcactttccgaac-3′

3′ primer, type II, AGRP-IMPDH; SEQ ID NO.: 59

5′-CGGACGACCAGCatatttcttcactttccgaac-3′

3′ primer, type II, NSPL-IMPDH; SEQ ID NO.: 60

5′-AAGCGGAGAGTTatatttcttcactttccgaac-3′

The 5′ primers included nucleotide sequences that are complementary to the N-terminal sequences of wild type impdh, which was modified to include an Nde I restriction site.

The 3′ primers included nucleotide sequences that encoded a substitute oligo-peptide (e.g., a tri- or tetra-peptide) and included sequences that are complementary to the Lys-Lys-Tyr at positions 108-110 of type II IMPDH, respectively.

The resulting amplified fragments were ligated with a cDNA fragment corresponding to the 820 bp C-terminal domain (Leu-244 to Phe-514) of the wild-type, type II impdh, to generate a DNA molecule encoding a modified type II, impdh polypeptide. The 820 bp fragment was generated by digestion of the wild type impdh DNA with Pvu II and Hind III restriction enzymes. The Pvu II restriction enzyme generated a blunt-ended cut, and cuts at the junction between the IMPDH subdomain and the C-terminal domain (e.g., Gln-243 and Leu-244), which provided a unique strategy for eliminating the subdomain (Glu-111 to Gln-243). The DNA molecule encoding a modified type II, IMPDH polypeptide was ligated into the pET24a vector (Novagen, Madison, Wis.).

The primer which includes the SPTQ tetra-peptide unexpectedly generated a modified IMPDH polypeptide having an SPT tri-peptide.

B) Isolating the Modified IMPDH Polypeptides

The pET24a vector includes the T7 RNA polymerase system which is inducible with IPTG. The recombinant vectors introduced into competent E. coli cells, BL21(DE3), (Novagen, Madison, Wis.) using a standard transformation procedure. The transformed cells were plated onto M9 minimal medium agar plates containing 100 μg/nl kanamycin, and incubated at 37° C. overnight. The individual colonies that grew on the M9 plates were selected, and inoculated into liquid M9 medium supplemented with 1% casamino acids, trace minerals, thiamine and vitamin B12, and containing 100 μg/mL kanamycin. The liquid cultures were grown overnight with vigorous shaking (e.g. 200 rpm) at 37° C.

The modified IMPDH polypeptides were expressed by culturing 0.5 liters of liquid M9 medium supplemented as described above in 2 liter baffled shake flasks, using the 10 mL overnight culture as an innoculant. The cells were grown at 37° C. with vigorous shaking until OD⁶⁰⁰=0.6-0.7, and IPTG was added to a final concentration of 0.5 mM. The cultures were then chilled on ice for 30 min, and grown for an additional 6 hours at 30° C. with vigorous shaking, and the cells were harvested by centrifugation at 10,000 RPM at 4° C. The cell pellets were frozen and stored at −80° C.

For large scale production of the modified IMPDH-DKT polypeptide, the procedure described above was followed. The scale was maintained at 0.5 L of liquid medium per 2 liter baffled shake flask. The cultures were initially grown at 37° C., followed by a 30 minute chilling period on ice, induction with 0.5 mM IPTG, and 6 hours post-induction growth at 30° C. These conditions reproducibly yielded 25-35 mg of the modified IMPDH-DKT polypeptide per liter of culture.

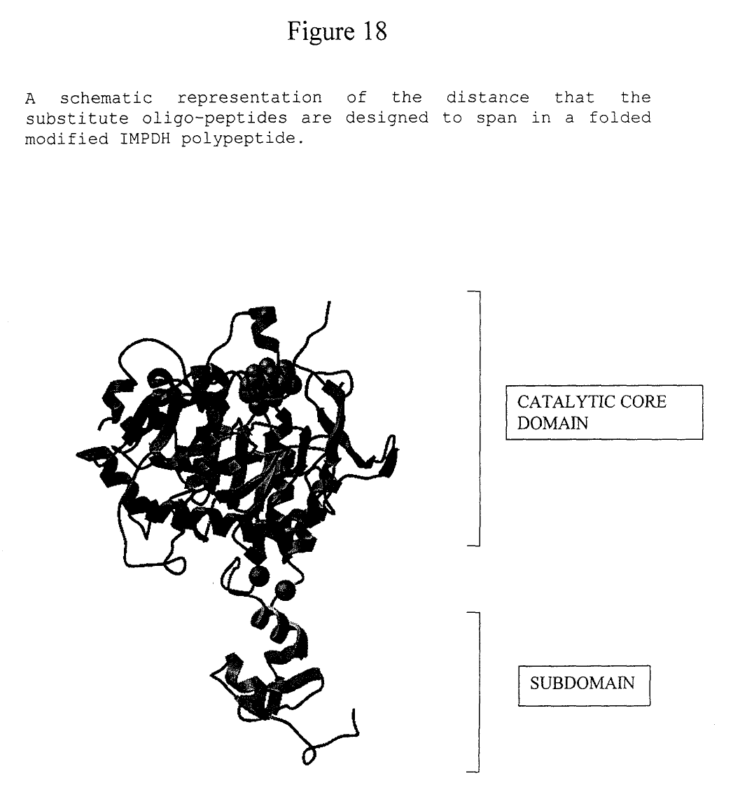

FIG. 18 illustrates a model of the wild type human type II, IMPDH protein. The catalytic core domain is located in the upper region, and the sub-domain is located in the lower region of the diagram. The two spheres located between the catalytic core and subdomain regions correspond to residues E-111 and Q-243. The distance between residues E-111 and Q-243 is approximately 5.1 Angstroms; the substitute oligo-peptides of the invention were designed to bridge this distance.

### Example 2

The following provides a description of the methods used to characterize the functional activity of the modified IMPDH polypeptides.

A) Isolating the Partially-purified Modified IMPDH Polypeptides

Polypeptides of the modified IMPDH-DKT, -SPS, -SPT, and -AGRP were isolated and partially purified using a blue dye affinity chromatographic step. All steps were performed at 4° C. unless otherwise noted. The frozen cell pellets (e.g., described in Example 1) were thawed on ice, and resuspended in 10 mL of Buffer A (25 mM Tris, pH 8.2, 20 mM KCl, 10% glycerol (v/v), 1 mM EDTA, 5 mM DTT, and 1 μg/mL leupeptin). The cells were lysed by 2×20 second sonication pulses using a ¼″ Microtip on a Branson model 450 sonifier (set on 2.5 power setting), and excessive foaming was carefully avoided. The samples were centrifuged for 20 min at 4° C. at 8500× g, and the supernatants were transferred to 15 mL tubes. To each sample was added 1.0 mL of 50% slurry containing Cibacron Blue 3GA dye resin (Sigma, product #8321), and the contents were mixed with gentle shaking for 2 hours. The samples were then transferred to individual 0.8×4.0 cm (Biorad, Poly-Prep, catalog number 731-1550), and the unbound material allowed to drain from the resins under gravity. Each column was washed with 6 mL Buffer A, then 6 mL Buffer B (e.g., Buffer A with 300 mM KCl), and finally 6 mL buffer C (e.g., Buffer A containing 1.5 M KCl), while collecting each 6 mL fraction in a clean tube. The samples were evaluated for the presence of the modified IMPDH polypeptides and to approximate the purity using standard SDS-PAGE methods, with Coomassie blue staining. For each of the modified IMPDH polypeptides, it was determined that the purity of the Buffer C fractions contained approximately 75% modified IMPDH polypeptide. The total protein content was quantitated using the Bradford assay method (Bradford, M. M., 1976, Anal. Biochem., 72: 248), with Biorad protein reagent (product #500-0006) and using BSA as a protein standard.

B) Detecting the Production of NADH

To evaluate the functional activity of the modified IMPDH polypeptides, the conversion of NAD to NADH was measured spectrophotometrically at 340 nm, 37° C. The assay buffer consisted of 50 mM Tris, pH 8.2 (37° C.), 100 mM KCl, 2 mM EDTA, and 3 mM DTT (i.e. buffer D) supplemented with 0.40 mM IMP and 0.40 mM NAD. Quartz cuvettes (1.0 cm pathlength) were used with each containing a total of 1.00 mL reaction mixture. Six concentrations of protein were evaluated simultaneously using a Cary Model 3E uv-vis spectrophotometer equipped with a multiple cell transport. Protein concentrations were varied between 20-300 nM. The production of NADH was determined by δOD³⁴⁰ (ε=6220 M⁻¹ cm⁻¹), measured for 5 min. The instrument software was used to calculate the initial rates of reaction as the linear least-squares fit to each data set.

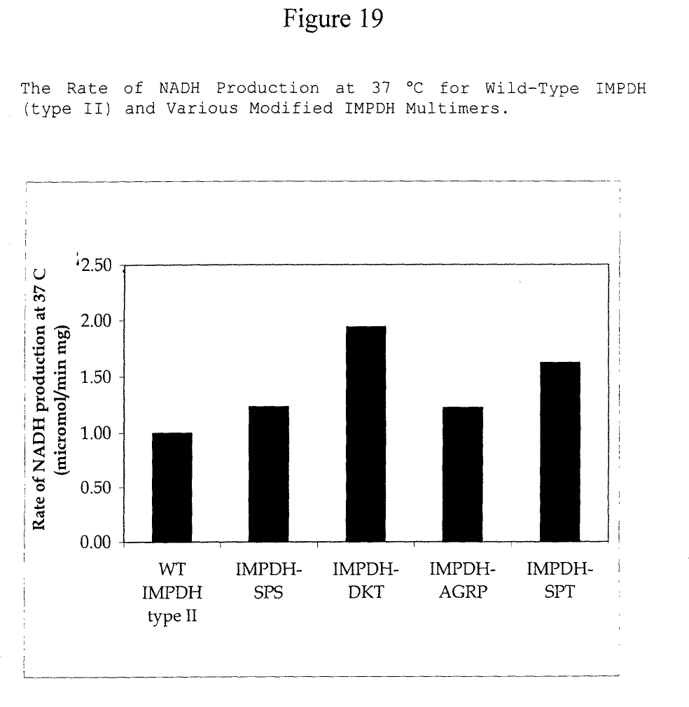

The modified IMPDH multimers -DKT, -SPS, -SPT, and -AGRP produced higher levels of NADH compared to the wild-type holoenzyme, producing levels that range from 1.23 to 1.94 μmoles/min mg protein. (FIG. 19) For example, multimers of IMPDH-DKT produced 1.94 μmoles/min mg protein, multimers of IMPDH-SPS produced 1.23 μmoles/min mg protein, multimers of IMPDH-SPT produced 1.62 μmoles/min mg protein, and multimers of IMPDH-AGRP produced 1.22 μmoles/min mg protein. In contrast, the wild-type IMPDH holoenzyme produced approximately 1.00 μmoles/min mg protein.

The modified IMPDH-DKT polypeptide multimer was analyzed further after purification to >95% purity. Kinetic characterization (e.g. based on kcat values) showed that the modified IMPDH-DKT polypeptide was nearly two-fold more active than wild type IMPDH type II.

### Example 3

The following provides a description of the methods used to evaluate the inhibitory effect of MPA on the functional activity of the modified IMPDH polypeptides.

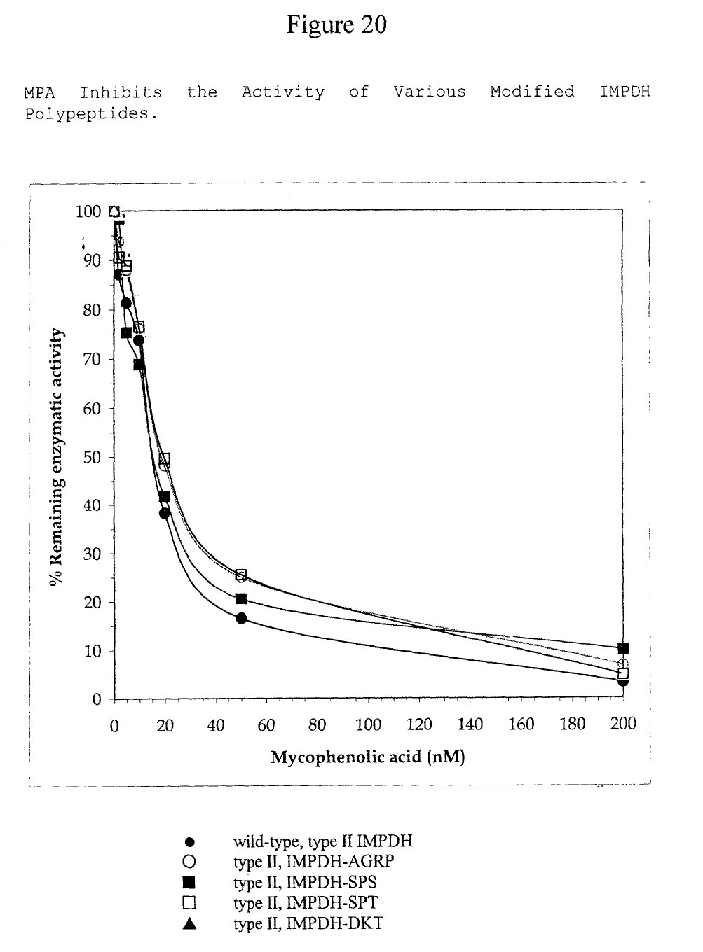

The inhibitory effect of MPA on the functional activity (e.g., NADH production) of the modified IMPDH multimers -DKT, -SPS, -SPT, and -AGRP was determined using a serial dilution method and a steady state enzyme kinetic method (FIG. 20) (S. F. Carr, et al. 1993 supra; B. Xiang, et al., 1996 supra).

The concentration of the modified IMPDH polypeptides was fixed at one concentration (e.g. 70 nM), and the inhibitor varied over 6 concentrations (i.e. 0, 2, 5, 10, 20, and 50 nM), under high substrate conditions (i.e. 0.40 mM IMP, 0.40 mM NAD) in buffer D. MPA (Sigma, product #M5255) was prepared in DMSO to 20 mM, and serial dilutions were prepared to allow 25 μL MPA sample per 1.00 mL reaction. The reactions were initiated by the addition of enzyme, and monitored for 15 min. The calculated initial rates were plotted as a function of MPA concentration, which demonstrated that each mutant was sensitive to MPA in the nanomolar range. Approximate IC₅₀ values were estimated by visual inspection of the normalized activity (i.e. no MPA present) versus MPA concentration plot, and the values ranged from 15-30 nM.

The activity of the modified IMPDH multimers and the wild-type human type II IMPDH holoenzyme were inhibited to 50% by MPA in the concentration range of 15-30 nM. For all samples, 3 μg of protein were used, corresponding to approximately 70 nM protein concentration for the modified IMPDH multimers and approximately 50 nM protein for the wild-type IMPDH holoenzyme.

The modified IMPDH-DKT polypeptide, purified to >95% purity, was further evaluated using steady state enzyme kinetic methods. Using a fixed IMPDH-DKT protein concentration of 50 nM, the substrates IMP and NAD were varied to measure initial velocities at 37° C., and to determine respective Kₘ and k_{cat} values. (Segel, I. H., 1975, Enzyme Kinetics: Behavior and Analysis of Rapid Equilibrium and Steady-state Enzyme Systems, John Wiley & Sons, New York, N.Y.). The following kinetic parameters were determined using buffer D, at 50 nM protein: k_{cat}, Kₘ^{IMP}, Kₘ^{NAD}, and Kᵢ^{XMP}. To determine Kᵢᵢ^{MPA}, 10 nM protein was used. The values were determined from global fits to replicate data sets using the appropriate models with either KinetAsyst kinetics software (IntelliKinetics, Princeton, N.J.) or The Scientist software (MicroMath® Scientific Software., Salt Lake City, Utah).

Steady state kinetic parameters

| Parameter | “DKT”-IMPDH-II | Inhibition type |
|---|---|---|
| k_{cat} | 2.14 ± .07 s⁻¹ | na |
| Kₘ^{IMP} | 20 ± 2 μM | na |
| Kₘ^{NAD} | 57 ± 3 μM | na |
| Kᵢ^{XMP} | 67 ± 5 μM | Competitive vs. IMP |
| Kᵢᵢ^{MPA} | 10 ± 3 nM | Uncompetitive vs. IMP |

### Example 4

The following provides a description of the methods used to obtain a purified sample of a modified IMPDH-DKT multimer and to obtain its X-ray crystal structure.

A) Purifying the Modified IMPDH Polypeptides

The IMPDH-DKT polypeptide was typically purified in two chromatographic steps to >95% purity. The frozen cells from 4 L of culture were thawed on ice in lysis buffer (buffer #1) consisting of 25 mM Tris, pH 8.2 (measured at 4° C.), 20 mM KCl, 10% glycerol (v/v), 2 mM EDTA, 5 mM DTT, 1 mM PMSF, 1 g/mL each bestatin, leupeptin, pepstatin, and E-64. All steps were performed at 4° C. unless noted otherwise. Cells were lysed on ice by sonication using a ¾″ probe tip and Branson model 450 sonifier, set on 7 power setting, and 2×5 minute cycles set at 30% duty cycle. The sample was centrifuged for 20 min at 8500× g, and the supernatant transferred to a clean polycarbonate bottle.

The protein was loaded onto a 50 mL column (2.5×10 cm) of Cibacron Blue 3GA fast flow agarose dye affinity resin (Sigma, catalog #C8321), equilibrated with buffer #2 (25 mM Tris, pH 8.2, 20 mM KCl, 10% glycerol (v/v), 1 mM EDTA, 5 mM DTT, and 1 μg/mL leupeptin). Protein was loaded with a peristaltic pump at 0.7 mL/min, and the flow monitored by absorbance detection at 280 nm. Unbound protein was eluted with buffer #2 at 3 mL/min until the absorbance returned to baseline. A KCl gradient was used over 15 column volumes to elute the IMPDH-DKT, using buffer #2 and buffer #3 (i.e. identical to buffer #2 except containing 2000 mM KCl). The IMPDH-DKT was identified by SDS-PAGE and activity assays (i.e. NADH production by δOD³⁴⁰), and suitable fractions were combined and dialyzed against 3 changes of buffer #4 (25 mM Tris, pH 8.2, 300 mM KCl, 10% glycerol (v/v), 1 mM EDTA, 5 mM DTT, and 1 μg/mL leupeptin).

IMP affinity chromatography was performed using 50 mL (2.5×10 cm) of resin prepared as described (i.e. Ikegami, T., et al, 1987, Life Sciences 40: 2277-2282). Protein was loaded onto the column at 0.7 mL/min, and washed with a total of 200 mL at 3 mL/min to remove unbound material. The IMPDH-DKT was specifically eluted at 3 mL/min using 200 mL of buffer #5 (25 mM Tris, pH 8.2, 300 mM KCl, 10% glycerol (v/v), 1 mM EDTA, 5 mM DTT, 2 mM IMP). The eluted protein was dialyzed against 4 changes of buffer #6 to remove unbound IMP (25 mM Tris, pH 8.2, 300 mM KCl, 10% glycerol (v/v), 1 mM EDTA, 5 mM DTT).

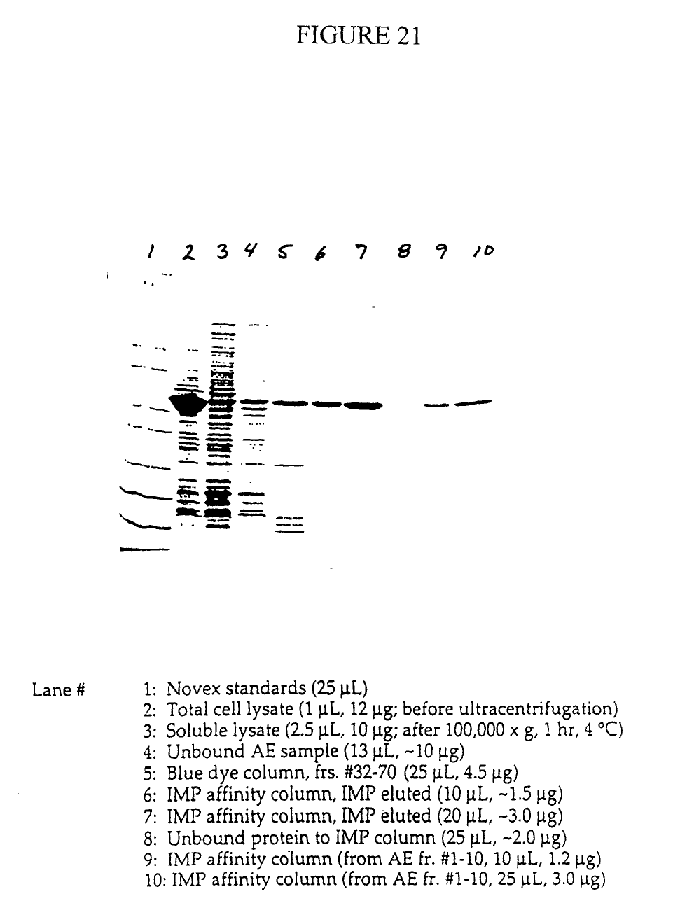

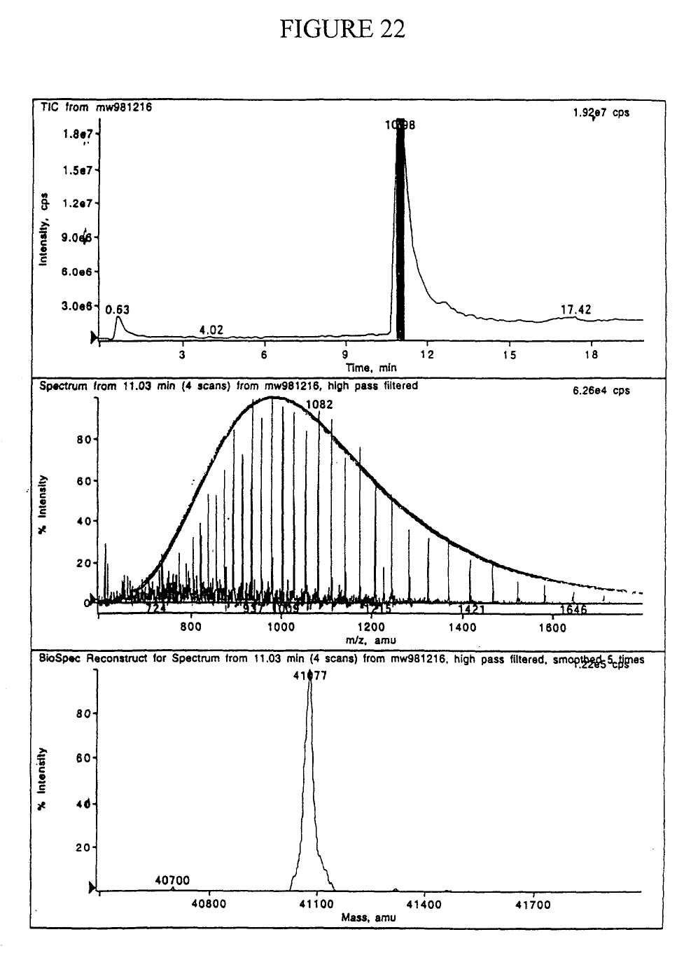

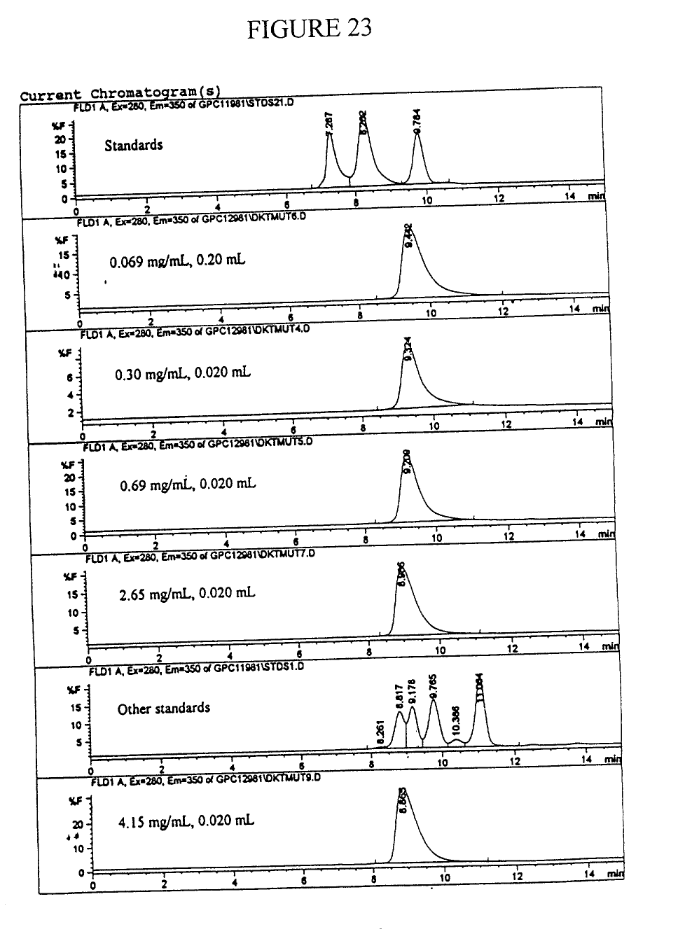

The purity of a sample of the IMPDH-DKT polypeptide is demonstrated in the Coomassie stained 4-20% Tris-glycine gel (FIG. 21), which shows a single band detected at approximately 42 kDa. The high degree of purity of this sample is also demonstrated in the accompanying HPLC-EMS trace (FIGS. 22A, B, C), and the analytical gel permeation chromatography trace (FIG. 23). The electrospray mass spectrometry identified a single protein component with mass consistent with the des-Met form of this modified with an observed mass of 41,077 Da (FIG. 22C). In addition, the results of the analytical gel permeation chromatography trace indicated that the modified IMPDH-DKT polypeptide is in dynamic equilibrium, probably between tetramer and octomer forms (FIG. 23). No aggregates were observed either by this method or by the dynamic light scattering method at 0.69 mg/mL. A UV-vis spectrum of the purified modified protein was consistent with a tightly bound nucleotide OD²⁸⁰/OD²⁶⁰=1.24, as was observed for wild-type IMPDH samples. This was not unexpected, since the final purification step involved elution from an IMP-affinity column (Ikegami, T., et al., 1987 Life Sciences 40:2277-2282). The results of circular dichroism spectrometry indicated the modified protein was well-folded and quite stable thermally (t_{½}˜75° C.).

### Example 5

The following provides a description of the methods used to obtain crystals of the modified IMPDH polypeptides.

A) Materials:

Abbreviations: IMPDH: inosine 5′-monophosphate dehydrogenase: IMP: inosine 5′-monophosphate: NAD⁺: β-nicotinamide adenine dinucleotide: MPA: mycophenolic acid: MEP: 1-methyl-2-pyrrolidinone: EDTA: ethylenediaminetetraacetic acid: DTT: dithiothreitol: ADA: N-[2-Acetamido]-2-iminodiacetic acid.

IMP (free acid), NAD⁺ (sodium salt), MPA, EDTA, ADA, Trizma base, KCl, and glycerol were purchased by Sigma Chemical Company. MEP, and DTT were purchased from Aldrich Chemical Company. Ultrafreeo-4 Centrifugal Filter & Tube Biomax-10K NMWL Membrane from Millipore.

B) Protein Crystallization:

The activity of the modified IMPDH-DKT was inhibited by the forward pathway (Fleming, M. A., et al., Biochemistry 35, 6990-6997). MPA was weighed and dissolved in MEP to a final concentration of 500 mM. Fresh buffer A (50 mM Tris-HCl, 300 mM KCl, 10% glycerol, 2 mM EDTA, 5 mM DTT, pH 8.0 at 23° C.) was prepared and filtered through a 0.22 μm filter. The purified protein was stored in buffer A at 0.69 mg/ml. NAD⁺, IMP, and MPA were then added in a two fold molar excess and allowed to equilibrate at room temperature for one hour. The inhibited complex was exhaustively concentrated and exchanged in fresh buffer A containing 2 mM MPA at 4° C. with a 10K NMWL membrane. Crystals of the IMPDH-DKT multimers were grown at room temperature using hanging drop vapor diffusion method. Two μl of protein solution at 2.97 mg/ml in buffer A containing 2 mM MPA was mixed with 2.0 μl of reservoir solution containing 11.75% saturated ammonium sulfate and 0.1 M ADA pH 6.5. The plate was sealed and allowed to equilibrate over 1.0 ml of reservoir. Crystals appeared within a few days and reached a maximum size of 0.09 mm×0.09 mm×0.07 mm in two weeks.

### Example 6

The following provides a description of the methods used to analyze the crystal structure of MPA bound to the modified IMPDH polypeptides.

The X-ray crystal structure of the modified IMPDH-DKT multimers was determined as follows. A crystal of IMPDH-DKT was co-crystallized with mycophenolic acid and was transferred to a solution containing 100 mM N-(2-Acetamido)-2-iminodiacetic acid, pH 6.5, 14% saturated ammonium sulfate, and 20% (v/v) glycerol as a cryoprotectant. The crystal was then lassoed with a fiber loop attached to a Hampton Research® cryo-pin and the loop and pin were plunged into liquid nitrogen (Rodgers, 1994). The crystal in the fiber loop was then mounted on a MAR® CCD 165 mm at IMCA beamline 171D at the Advanced Photon Source at Argonne National Laboratory and data were collected at a wavelength of 1 Angstrom. The space group was 1422 with a=b=102.9 Angstrom; c=178.3 Angstrom. The data were integrated and reduced with the HKL suite of programs (Otwinowski, Z., & Minor, W., 1997 Methods in Enzymology 276:307-326). Statistics of the data collection are summarized below:

| | | | | % | | |
|---|---|---|---|---|---|---|
| | Resolution | | | Com- | | |
| | (Angstrom) | Measured | Unique | plete | R-sym | I/σ(I) |
| Overall | 20-2.2 | 195663 | 24616 | 99.5 | 0.072 | 32.0 |
| Last Shell | 2.28-2.2 | ≧17096 | 2417 | 99.9 | 0.296 | 7.4 |

The resolution of the data was limited by the rather small aperture of the detector. Later, data was also collected using CuKα (1.54 Angstrom) radiation to 2 Angstrom resolution.

The structure of the IMPDH-DKT mutant was determined by molecular replacement (Rossmann, M. G., 1990 Acta Crystallogr. Sect. A 46:73-82) using the AmoRe program (Navaza, J., 1994 Acta Crystallogr. Sect. A 50:157-163) as implemented in the CCP4 program suite (Collaborative Computational Project, Number 4, 1994 Acta Cystallogr. Sect. D 50, 760-763). The model consisted of residues 17-110, 244-420 and 427-514 of hamster IMPDH II (Sinchak, M. D., et al., 1996 Cell 85, 921-930). Due to sequence differences between hamster and human the following residues were reduced to Ala: 265, 290, 292, and residue 327 converted from Cys to Ser. Data were used from 10-4 Angstrom resolution in both the rotation and translation function. The rotation function solution had a signal-to-noise ratio of 1.9 and the translation function solution had a signal-to-noise ratio of 1.8, both indicating a very clean structure determination. After least-squares rigid body fitting the correlation coefficient was 70.8 and the R-value was 32.8, again indicating a very clean structure determination. The structure was refined with X-PLOR (Brünger, A. T., 1992 X-PLOR version 3.1, Yale University Press, New Haven) and manual re-building was accomplished with CHAIN (Sack, J. S., 1988 J. Mol. Graph. 6:224-225). The final model consisted of 2684 protein atoms, inosine monophosphate covalently bound to Cys 331, mycophenolic acid and 219 solvent molecules. The R-value was 0.201 and the free R-value was 0.264 for data from 8-2.2 Å resolution with r.m.s. deviation from ideal bond lengths of 0.011 Angstrom, ideal bond angles of 1.5°, and improper dihedral angles of 1.5°.

This structure showed that the IMPDH-DKT multimer bound IMP and mycophenolic acid in exactly the same manner as shown in the earlier work on intact hamster IMPDH (Sinchak, M. D., et al., 1996 Cell 85, 921-930). Due to higher resolution, this structure was of higher quality as evidenced by the ability to identify an unusual cis peptide bond in the vicinity of the active site between Gly 302 and Asn 303. Moreover, the coordinates of hamster IMPDH II provided to us by Vertex, did not include solvent molecules, although they were described in the paper. (Sinchak, M. D., et al., 1996 Cell 85, 921-930). The results disclosed herein provided the sites of water molecules in the active site, which were extremely valuable for modeling. Additionally, six residues at the N-terminus (numbers 11-16) as well as the DKT insert (numbered 110A, 110B and 110C, respectively) and residue 421 main chain could be fitted to electron density that were not in the model of the hamster structure.

In summary, the modified IMPDH-DKT multimer is an excellent subject for structure-based drug design. The molecule with mycophenolic acid was easily crystallized and yielded crystals that diffracted to 2 Angstrom resolution in the laboratory, well within the range to provide high-quality information to chemists and molecular modelers for the design of more potent inhibitors and inhibitors with more desirable properties.

What is claimed is:

1. An isolated, modified IMPDH polypeptide comprising an oligo-peptide domain substituted for a subdomain of a wild-type IMPDH polypeptide, the substitution resulting in a modified IMPDH polypeptide, which is shorter in length compared to the wild-type IMPDH polypeptide.

2. The modified IMPDH polypeptide of claim 1, wherein the wild type IMPDH polypeptide is type I or type II IMPDH.

3. The modified IMPDH polypeptide of claim 1, further comprising a first IMPDH catalytic core domain and a second IMPDH catalytic core domain.

4. The isolated, modified IMPDH polypeptide of claim 3, wherein the oligo-peptide domain is located between the first and the second IMPDH catalytic core domains.

5. The isolated, modified IMPDH polypeptide of claim 3, wherein the first IMPDH catalytic core domain is located N-terminal to the second IMPDH catalytic core domain.

6. The modified IMPDH polypeptide of claim 1, wherein the oligo-peptide domain comprises a tri-peptide.

7. The modified IMPDH polypeptide of claim 1, wherein the oligo-peptide domain comprises a tetra-peptide.

8. The modified IMPDH polypeptide of claim 1 having the amino acid sequence as shown in any one of SEQ ID NOS.: 20-39.

9. The modified IMPDH polypeptide of claim 6, wherein the tri-peptide has an amino acid sequence as shown in any one of SEQ ID NOS.: 1-10.

10. The modified IMPDH polypeptide of claim 7, wherein the tetra-peptide has an amino acid sequence as shown in any one of SEQ ID NOS.:11-19.

11. The isolated, modified IMPDH polypeptide of claim 6, wherein the first amino acid position of the tri-peptide sequence is selected from the group consisting of aspartic acid, threonine, serine, or glycine, lysine, isoleucine and alanine.

12. The isolated modified IMPDH polypeptide of claim 6, wherein the second amino acid position of the tri-peptide sequence is selected from the group consisting of lysine, proline, alanine, valine, leucine, glycine and serine.

13. The isolated modified IMPDH polypeptide of claim 6, wherein the third amino acid position of the tri-peptide sequence is selected from the group consisting of tyrosine, serine, threonine, glycine, phenylalanine, isoleucine, histidine, and aspartic acid.

14. The isolated modified IMPDH polypeptide of claim 7, wherein the first amino acid position of the substitute tetra-peptide sequence is selected from the group consisting of glycine, glutamine, asparagine, serine, threonine, tyrosine, and alanine.

15. The isolated modified IMPDH polypeptide of claim 7, wherein the second amino acid position of the substitute tetra-peptide sequence is selected from the group consisting of serine, glycine, pro line, iso leucine, and arginine.

16. The isolated modified IMPDH polypeptide of claim 7, wherein the third amino acid position of the substitute tetra-peptide sequence is selected from the group consisting of serine, glutamine, threonine, tyrosine, isoleucine, proline, and arginine.

17. The isolated modified IMPDH polypeptide of claim 7, wherein the fourth amino acid position of the substitute tetra-peptide sequence is selected from the group consisting of tryptophan, proline, leucine, serine, glutamine, threonine, and tyrosine.

18. A protein multimer, comprising between 1 and 8 modified IMPDH polypeptides in association with each other, wherein the modified IMPDH polypeptides each comprise an oligo-peptide domain substituted for a subdomain of a wild-type IMPDH polypeptide resulting in the modified IMPDH polypeptide, which is shorter in length compared to the wild-type IMPDH polypeptide.

19. The protein multimer of claim 18 which is a dimer.

20. The protein multimer of claim 18 which is a tetramer.

21. The protein multimer of claim 18 which is an octamer.

22. A nucleic acid molecule comprising a polynucleotide sequence which encodes any one of the modified IMPDH polypeptides of claim 8.

23. The nucleic acid molecule of claim 22 which is RNA.

24. The nucleic acid molecule of claim 22 which is DNA.

25. A nucleic acid molecule comprising a polynucleotide sequence which is complementary to the polynucleotide sequence of claim 24.

26. The nucleic acid molecule of claim 22, 23, 24 or 25 which is labeled with a detectable marker.

27. The nucleic acid molecule of claim 26, wherein the detectable marker is selected from the group consisting of a radioisotope, a fluorescent compound, a bioluminescent compound, a chemiluminescent compound, a metal chelator, and an enzyme.

28. A vector comprising a polynucleotide sequence which encodes any one of the modified IMPDH polypeptides of claim 8.

29. A host-vector system comprising the vector of claim 28 in a suitable host cell.

30. The host-vector system of claim 29, wherein the suitable host cell is from an organism which is selected from the group consisting of bacteria, yeast, mammals, insects, and plants.

31. A method for producing a modified IMPDH polypeptide comprising:
a) culturing the host-vector system of claim 30 under suitable conditions so as to produce the modified IMPDH polypeptide; and
b) recovering the modified IMPDH polypeptide so produced.

32. The modified IMPDH polypeptide produced by the method of claim 31.

33. A monoclonal antibody reactive with the modified IMPDH polypeptide of claim 1 or 32.

34. The monoclonal antibody of claim 33 which is labeled with a detectable marker.

35. The monoclonal antibody of claim 34, wherein the detectable marker is selected from the group consisting of a radioisotope, a fluorescent compound, a bioluminescent compound, a chemiluminescent compound, a metal chelator, and an enzyme.

36. A method for identifying an agent of interest that inhibits the activity of a protein multimer comprising modified IMPDH polypeptides, the method comprising:
a) contacting the protein multimer with inosine-5′-monophosphate, nicotinamide adenine dinucleotide, and the agent of interest; and
b) detecting the level of the reduced form of inosine-5′-monophosphate or nicotinamide adenine dinucleotide which is generated, whereby a low level of the reduced form of inosine-5′-monophosphate or nicotinamide adenine dinucleotide generated indicates that the agent of interest inhibits IMPDH activity.

37. The method according to claim 36 which comprises contacting a plurality of substantially identical samples each separately with a different agent of interest.

38. The method of claim 37, wherein the plurality of samples comprises more than about 10⁴ samples.

39. The method of claim 37, wherein the plurality of samples comprises more than about 10⁵ samples.

40. The method of claim 37, wherein the plurality of samples comprises more than about 10⁶ samples.

41. The method of claim 40, wherein the plurality of substantially identical samples are each contacted essentially simultaneously with a different agent of interest.

42. A method for improving resolution of an X-ray crystal structure of an IMPDH polypeptide or IMPDH polypeptide complex comprising removing a subdomain of the IMPDH polypeptide thereby improving the resolution of the X-ray crystal structure of the IMPDH polypeptide or IMPDH polypeptide.

43. The method of claim 42, wherein the IMPDH polypeptide has the amino acid sequence of any one of SEQ ID NOs:20-39.

44. The method of claim 42, wherein the protein or protein complex is complexed with a compound.

45. The method of claim 44, wherein the compound is an inhibitor.

46. The method of claim 45, wherein the inhibitor is MPA.

47. The method of claim 42, wherein the subdomain of the IMPDH polypeptide is replaced with a relatively shorter peptide fragment.

48. A method for improving resolution of an X-ray crystal structure of an IMPDH polypeptide comprising reducing the length of the IMPDH polypeptide thereby improving the resolution of the X-ray crystal structure of the IMPDH polypeptide.

49. A method for improving resolution of an X-ray crystal structure of an IMPDH polypeptide comprising substituting amino acids 111-243 of any one of SEQ ID NOS: 48, 49, 62, 63, 64, or 65 with a tri-peptide thereby improving the resolution of the X-ray crystal structure of the IMPDH polypeptide.

50. The method of claim 49, where in the tri-peptide has an amino acid sequence as shown in any one of SEQ ID NOS.:1-10.

51. A method for improving resolution of an X-ray crystal structure of an IMPDH polypeptide comprising substituting amino acids 111-243 of any one of SEQ ID NOS: 48, 49, 62, 63, 64, or 65 with a tetra-peptide thereby improving the resolution of the X-ray crystal structure of the IMPDH polypeptide.

52. The method of claim 51, wherein the tetra-peptide has an amino acid sequence as shown in SEQ ID NOs: 11-19.

53. A modified IMPDH polypeptide comprising an amino acid sequence as shown in any one of SEQ ID NOS.:20-39.

54. An isolated nucleic acid molecule comprising the nucleic acid sequence as shown in any one of SEQ ID NOS.:40-47.

55. A modified IMPDH polypeptide, wherein amino acids 111-243 as shown in any one of SEQ ID NOS:48, 49, 62, 63, 64, or 65 are replaced with a tripeptide.

56. A modifified IMPDH polypeptide, wherein amino acids 111-243 as shown in any one of SEQ ID NOS:48, 49, 62, 63, 64, or 65 are replaced with a tetrapeptide.
